SEQUENCE LISTING (10 sequences)

SEQ ID NO: 1 - protein, 390 aa, synthetic construct
  REGION 1..390: note = Synthetic: TGF-B polypeptide
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 2 - protein, 112 aa, synthetic construct
  REGION 1..112: note = Synthetic: TGF-B polypeptide
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 3 - protein, 414 aa, synthetic construct
  REGION 1..414: note = Synthetic: TGF-B polypeptide
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 4 - protein, 112 aa, synthetic construct
  REGION 1..112: note = Synthetic: TGF-B polypeptide
ALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 5 - protein, 412 aa, synthetic construct
  REGION 1..412: note = Synthetic: TGF-B polypeptide
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 6 - protein, 112 aa, synthetic construct [sequence omitted]

SEQ ID NO: 7 - protein, 169 aa, synthetic construct
  REGION 1..169: note = Synthetic: IL-2 polypeptide
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 8 - protein, 149 aa, synthetic construct
  REGION 1..149: note = Synthetic: IL-2 polypeptide
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 9 - protein, 153 aa, synthetic construct
  REGION 1..153: note = Synthetic: IL-2 polypeptide
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 10 - protein, 133 aa, synthetic construct [sequence omitted]

(19) United States
(12) Patent Application Publication
(10) Pub. No.: US 2024/0293509 A1
(43) Pub. Date: Sep. 5, 2024

(54) METHODS OF TREATING INFLAMMATORY BOWEL DISEASE

(71) Applicant: UNIVERSITY OF PITTSBURGH - OF THE COMMONWEALTH SYSTEM OF HIGHER EDUCATION, Pittsburgh, PA (US)

(72) Inventors: Roger Sands, Sewickley, PA (US); Steven R. Little, Allison Park, PA (US)

(73) Assignee: UNIVERSITY OF PITTSBURGH - OF THE COMMONWEALTH SYSTEM OF HIGHER EDUCATION, Pittsburgh, PA (US)

(21) Appl. No.: 18/640,662

(22) Filed: Apr. 19, 2024

Related U.S. Application Data
(63) Continuation of application No. PCT/US2022/047292, filed on Oct. 20, 2022.
(60) Provisional application No. 63/257,804, filed on Oct. 20, 2021.

(51) Int. Cl. A61K 38/18; A61K 9/16; A61K 31/436; A61K 38/20; A61P 1/04

(52) U.S. Cl. CPC A61K 38/1841; A61K 9/1647; A61K 31/436; A61K 38/2013; A61P 1/04

(57) ABSTRACT

The present disclosure relates to the use of microparticles to prevent and/or treat inflammatory bowel disease (IBD). The methods include prevention and/or treatment of IBD with the administration of the disclosed microparticles to a subject. The present disclosure further provides kits for performing such methods.

### CROSS-REFERENCE TO RELATED APPLICATIONS

This application is a continuation of International Patent Application No. PCT/US2022/047292, filed on Oct. 20, 2022, which claims priority to U.S. Provisional Patent Application Ser. No. 63/257,804, filed on Oct. 20, 2021, the contents of each of which are hereby incorporated by reference in their entireties, and to each of which priority is claimed.

### STATEMENT OF GOVERNMENT INTEREST

This invention was made with government support under grant number DK063922 awarded by the National Institutes of Health. The government has certain rights in the invention.

### SEQUENCE LISTING

This application contains a Sequence Listing, which has been submitted in XML format via EFS-Web and is hereby incorporated by reference in its entirety. Said XML copy, created on Apr. 19, 2024, is named 072396_1009_SLST26.xml and is 15,672 bytes in size.

### FIELD OF INVENTION

The present disclosure relates to the use of microparticles to prevent and/or treat Inflammatory Bowel Disease (IBD).

### BACKGROUND

Inflammatory bowel disease (IBD) affects approximately 2 μmillion people in the United States alone and contributes to significant morbidity with many patients not responding to medical therapy and approximately 40% of patients eventually requiring surgery (Kappelman et al., Clinical Gastroenterology and Hepatology 5, 1424-1429 (2007); Molodecky et al., Gastroenterology 142, 46-54 (2012); Ganz et al., Inflammatory Bowel Diseases 22, 1032-1041 (2016)). IBD is a chronic, lifelong disease with a peak incidence between the ages of 15 and 30 with a growing prevalence and incidence particularly in developing countries. In addition to significant physical and emotional costs, the direct costs of Crohn's Disease and ulcerative colitis, the two most common forms of inflammatory bowel disease, range from 11-28 billion dollars per year in the United States alone (C. s. a. C. Foundation, The Facts about Inflammatory Bowel Diseases. (2014)). Exemplifying the significant economic costs of the disease, Humira, an anti-tumor necrosis factor biologic commonly used in inflammatory bowel disease and rheumatologic conditions, had 18 billion dollars of world-wide revenue in 2017 (AbbVie, AbbVie Reports Full-Year and Fourth-Quarter 2017 Financial Results. (2018)).

Despite the significant costs of the disease, no cure exists and despite recent improvements in biologic therapies, more than ⅓ of patients do not respond to medication or lose responsiveness to therapy (Roda et al., Clinical and Translational Gastroenterology 7, (2016)). In addition to being ineffective in these cases, the side effect profile is significant, and the medications are often expensive (Verstockt et al., J. Gastroenterol. 53, 585-590 (2018)). Specifically, the majority of therapies that do exist are administered systemically and increase the risk for off target adverse effects including an increased risk of cancer and infection. Further, most of the therapies target downstream cytokines rather than the upstream effector cells including regulatory T cells and effector T cells. Regulatory T cells play an important role for dampening immunity in many diseases of inappropriate immune activation including inflammatory bowel disease. However, current pharmacotherapy does not directly target regulatory T cells and ex vivo administration, despite promising early results, is laborious and costly. Additionally, many of the most potent and efficacious therapies, the biologics, are administered subcutaneously or intravenously, therefore reducing patient compliance compared to oral therapeutics. As such, new approaches are needed to treat individuals that do not respond to traditional therapy with potent therapeutics that can be administered orally.

### SUMMARY

The present disclosure provides compositions, kits and methods for preventing or treating a gastrointestinal condition. In certain embodiments, the present disclosure provides a method for treating a gastrointestinal condition in a subject in need thereof. In certain embodiments, the method comprises administering a composition comprising a first microparticle comprising a transforming growth factor beta (TGF-β) polypeptide. In certain embodiments, the composition further comprises a second microparticle comprising an interleukin. In certain embodiments, the composition comprises a third microparticle comprising a macrolide. In certain embodiments, the method for treating a gastrointestinal condition comprises a composition formulated for oral administration. In certain embodiments, the first, second, and third microparticles are controlled release microparticles.

In certain embodiments, the gastrointestinal condition is selected from the group consisting of inflammatory bowel disease (IBD), gastritis, peptic ulcers, esophagitis, cholecystitis, Gastro-Intestinal Graft Versus Host Disease (GI-GVHD), gastrointestinal cancers or tumors, gastrointestinal infections, and gastrointestinal immunopathies. In certain embodiments, the IBD is selected from the group consisting of Crohn's Disease or ulcerative colitis.

In certain embodiments, the first, second, and third microparticles comprise a polymer. In certain embodiments, the first, second, and third microparticles comprise the same polymer.

In certain embodiments, the first microparticle comprises a poly(ethylene glycol) polymer, poly(lactic acid) polymer, poly(glycolic acid) polymer, poly(lactide-co-glycolide) polymer, polycaprolactone polymer, or a combination thereof. In certain embodiments, the second microparticle comprises a poly(ethylene glycol) polymer, poly(lactic acid) polymer, poly(glycolic acid) polymer, poly(lactide-co-glycolide) polymer, polycaprolactone polymer, or a combination thereof. In certain embodiments, the third microparticle comprises a poly(ethylene glycol) polymer, poly(lactic acid) polymer, poly(glycolic acid) polymer, poly(lactide-co-glycolide) polymer, polycaprolactone polymer, or a combination thereof. In certain embodiments, the first, second, and third microparticles comprise a poly(lactide-co-glycolide) polymer. In certain embodiments, the first, second, and third microparticles have a diameter between about 5 μm to about 25 μm in diameter.

In certain embodiments, the composition is a hydrogel composition. In certain embodiments, the hydrogel composition comprises alginate. In certain embodiments, the hydrogel composition comprises chitosan.

In certain embodiments, the transforming growth factor beta (TGF-β) polypeptide is a TGF-β-1 polypeptide. In certain embodiments, the TGF-β polypeptide comprises an amino acid sequence that is at least about 80% identical to the amino acid sequence set forth in SEQ ID NO: 1. In certain embodiments, the TGF-β polypeptide comprises the amino acid sequence set forth in SEQ ID NO: 1. In certain embodiments, the TGF-β polypeptide comprises an amino acid sequence that is at least about 80% identical to the amino acid sequence set forth in SEQ ID NO: 2. In certain embodiments, the TGF-β polypeptide comprises the amino acid sequence set forth in SEQ ID NO: 2. In certain embodiments, the TGF-β polypeptide comprises two copies of the amino acid sequence set forth in SEQ ID NO: 2. In certain embodiments, the two copies are linked by a disulfide bond. In certain embodiments, a weight ratio of the TGF-β polypeptide to the polymer of the first microparticle is between about 1:100000 and about 1:1.

In certain embodiments, the interleukin polypeptide is IL-2. In certain embodiments, the interleukin polypeptide comprises an amino acid sequence that is at least about 80% identical to the amino acid sequence set forth in SEQ ID NO: 9. In certain embodiments, the interleukin polypeptide comprises the amino acid sequence set forth in SEQ ID NO: 9. In certain embodiments, the interleukin polypeptide comprises an amino acid sequence that is at least about 80% identical to the amino acid sequence set forth in SEQ ID NO: 10. In certain embodiments, the interleukin polypeptide comprises the amino acid sequence set forth in SEQ ID NO: 10. In certain embodiments, a weight ratio of the interleukin polypeptide to the polymer of the second microparticle is between about 1:100000 and about 1:1.

In certain embodiments, the macrolide is an mTOR inhibitor. In certain embodiments, the macrolide is rapamycin. In certain embodiments, a weight ratio of the macrolide to the polymer of the third microparticle is between about 1:100000 and about 1:1.

In certain embodiments, the present disclosure provides for a method comprising administering a composition comprising a first microparticle comprising a transforming growth factor beta 1 (TGF-β1) polypeptide, a second microparticle comprising an interleukin-2 (IL-2) polypeptide, and a third microparticle comprising a rapamycin.

In certain embodiments, the present disclosure provides for a method comprising administering a composition comprising at least one microparticle. In certain embodiments the at least one microparticle is selected from the group consisting of a microparticle comprising a transforming growth factor beta (TGF-β) polypeptide, a microparticle comprising an interleukin polypeptide, a microparticle comprising a macrolide, and combinations thereof.

The present disclosure further provides a composition for use in the treatment of a gastrointestinal condition. In certain embodiments, the composition comprises a first microparticle comprising a transforming growth factor beta (TGF-β) polypeptide; a second microparticle comprising an interleukin polypeptide; and a third microparticle comprising a macrolide. In certain embodiments, the first, second, and third microparticles are controlled release microparticles. In certain embodiments, the first, second, and third microparticles comprise a polymer. In certain embodiments, the first, second, and third microparticles comprise the same polymer. In certain embodiments, the composition is formulated for oral administration to a subject.

In certain embodiment, the composition is for use in the treatment of a gastrointestinal condition selected from the group consisting of inflammatory bowel disease (IBD), gastritis, peptic ulcers, esophagitis, cholecystitis, Gastro-Intestinal Graft Versus Host Disease (GI-GVHD), gastrointestinal cancers or tumors, gastrointestinal infections, and gastrointestinal immunopathies. In certain embodiments, the IBD is selected from the group consisting of Crohn's Disease or ulcerative colitis.

In certain embodiments, the first microparticle comprises a poly(ethylene glycol) polymer, poly(lactic acid) polymer, poly(glycolic acid) polymer, poly(lactide-co-glycolide) polymer, polycaprolactone polymer, or a combination thereof. In certain embodiments, the second microparticle comprises a poly(ethylene glycol) polymer, poly(lactic acid) polymer, poly(glycolic acid) polymer, poly(lactide-co-glycolide) polymer, polycaprolactone polymer, or a combination thereof. In certain embodiments, the third microparticle comprises a poly(ethylene glycol) polymer, poly(lactic acid) polymer, poly(glycolic acid) polymer, poly(lactide-co-glycolide) polymer, polycaprolactone polymer, or a combination thereof. In certain embodiments, the first, second, and third microparticles comprise a poly(lactide-co-glycolide) polymer. In certain embodiments, the first, second, and third microparticles have a diameter between about 5 μm to about 25 μm in diameter.

In certain embodiments, the composition is a hydrogel composition. In certain embodiments, the hydrogel composition comprises alginate. In certain embodiments, the hydrogel composition comprises chitosan.

In certain embodiments, the transforming growth factor beta (TGF-β) polypeptide is a TGF-β-1 polypeptide. In certain embodiments, the TGF-β polypeptide comprises an amino acid sequence that is at least about 80% identical to the amino acid sequence set forth in SEQ ID NO: 1. In certain embodiments, the TGF-β polypeptide comprises the amino acid sequence set forth in SEQ ID NO: 1. In certain embodiments, the TGF-β polypeptide comprises an amino acid sequence that is at least about 80% identical to the amino acid sequence set forth in SEQ ID NO: 2. In certain embodiments, the TGF-β polypeptide comprises the amino acid sequence set forth in SEQ ID NO: 2. In certain embodiments, the TGF-β polypeptide comprises two copies of the amino acid sequence set forth in SEQ ID NO: 2. In certain embodiments, the two copies are linked by a disulfide bond. In certain embodiments, a weight ratio of the TGF-β polypeptide to the polymer of the first microparticle is between about 1:100000 and about 1:1.

In certain embodiments, the interleukin polypeptide is IL-2. In certain embodiments, the interleukin polypeptide comprises an amino acid sequence that is at least about 80% identical to the amino acid sequence set forth in SEQ ID NO: 9. In certain embodiments, the interleukin polypeptide comprises the amino acid sequence set forth in SEQ ID NO: 9. In certain embodiments, the interleukin polypeptide comprises an amino acid sequence that is at least about 80% identical to the amino acid sequence set forth in SEQ ID NO: 10. In certain embodiments, the interleukin polypeptide comprises the amino acid sequence set forth in SEQ ID NO: 10. In certain embodiments, a weight ratio of the interleukin polypeptide to the polymer of the second microparticle is between about 1:100000 and about 1:1.

In certain embodiments, the macrolide is an mTOR inhibitor. In certain embodiments, the macrolide is rapamycin. In certain embodiments, a weight ratio of the macrolide to the polymer of the third microparticle is between about 1:100000 and about 1:1.

In certain embodiments, the present disclosure provides for a composition comprising a first microparticle comprising a transforming growth factor beta 1 (TGF-β1) polypeptide, a second microparticle comprising an interleukin-2 (IL-2) polypeptide, and a third microparticle comprising a rapamycin.

In certain embodiments, the present disclosure provides a composition comprising at least one microparticle. In certain embodiments the at least one microparticle is selected from the group consisting of a microparticle comprising a transforming growth factor beta 1 (TGF-β) polypeptide, a microparticle comprising an interleukin-2 (IL-2) polypeptide, a microparticle comprising a macrolide, and combinations thereof.

### BRIEF DESCRIPTION OF THE DRAWINGS

FIG. 1 shows a schematic of the intersection of the IBD interactome with TGF-β, IL-2, and rapamycin signaling. Many of the proteins involved in IBD pathogenesis are affected by TGF-β, IL-2 or rapamycin. The proteins implicated in IBD pathogenesis as identified using Ingenuity Pathway Analysis were plotted circumferentially along with TGF-β, IL-2, and rapamycin. Proteins involved in TGF-β, IL-2, and rapamycin signaling within the Ingenuity Pathway Analysis databank that are also found in the IBD pathogenesis network are depicted as chords.

FIGS. 2A-2E show poly(lactide-co-glycolide) microparticles for the controlled release of TGF-β, IL-2, and rapamycin. Microparticles were synthesized by either water-in-oil or water-in-oil-in-water emulsions. FIG. 2A shows a representative scanning electron micrograph (SEM) image of presently disclosed microparticles. FIG. 2B shows distribution of microparticle size measured in microns. Poly(lactide-co-glycolide) microparticles were used to deliver TGF-β, IL-2, and rapamycin. FIGS. 2C-2E show release of the factors (TGF-3, IL-2, and rapamycin) for 1 week measured by ELISA assay. FIG. 2C shows microparticle release of TGF-β. FIG. 2D shows microparticle release of IL-2. FIG. 2E shows microparticle release of rapamycin.

FIGS. 3A-3D show the effects of therapeutic administration of microparticles containing TGF-β, IL-2, and rapamycin in a murine model of inflammatory bowel disease. Microparticles containing IL-2, TGF-β, and rapamycin attenuated disease severity when administered orally in a murine model of inflammatory bowel disease. Three (3) days after disease induction, animals were left untreated (TNBS) or treated with soluble factors, blank microparticles, or microparticles containing IL-2, TGF-β, or rapamycin. FIG. 3A shows a schematic outlining the experimental approach with animals receiving a single treatment.

FIG. 3B shows the survival curves of mice after receiving a single treatment. Naïve animals were not treated throughout the experiment. FIG. 3C shows a schematic outlining a second set of experiments, where animals were treated on days 1 and 3 or left untreated (TNBS).

FIG. 3D shows the survival curves of mice after receiving two (2) treatments. In FIGS. 3A and 3B: n=4 for naïve, untreated animals and for the other conditions n=7-8 μmice per condition; in FIGS. 3C and 3D: n=26 for TNBS treated animals and n=15 for the orally administered trifactor microparticles (PO TRI MP). Statistical analysis was completed using a one-way log-rank test and log-rank hazard ratio.

FIGS. 4A-4E show the histopathology analysis following therapeutic administration of trifactor microparticles (TRI MP) in a murine model of inflammatory bowel disease (IBD). Mice with colitis receiving TRI MP had attenuated colitis compared to experimental controls. FIG. 4A shows colitis scores at day 7 following disease induction. FIG. 4B shows colon measurements prior to pathologic examination. Blinded selected representative pathologic images are shown in FIGS. 4C, 4D and 4E. FIG. 4C shows a representative image of naïve colon tissue. FIG. 4D shows a representative image of pathologic tissue left untreated. FIG. 4E shows a representative image of pathological tissue from a mouse receiving TRI MP. Statistical analysis was completed using a Kruskal-Wallis test with Dunn's multiple comparison.

FIGS. 5A and 5B show therapeutic administration of trifactor microparticles (TRI MP)compared to controls in a murine model of inflammatory bowel disease. FIG. 5A shows a schematic outlining the experimental design for microparticle therapeutic delivery in mice. Three days after disease induction, animals were treated with soluble factors, blank microparticles, or TRI MP (PR: per rectum, PO: per os). n=8-9 μmice per control group. FIG. 5B shows the survival curves of mice after receiving treatment. IL-2, TGF-β, and rapamycin when administered as a bolus promoted disease severity while rectal administration had little effect and blank microparticles had a nonsignificant trend toward worse disease outcomes in comparison to TRI MP delivery. Statistical analysis was completed using a log-rank test and log-rank hazard ratio corrected with Bonferroni for multiple comparisons.

FIGS. 6A and 6B show adherence of fluorescently labelled microparticles to naïve and inflamed murine colonic tissue. FIG. 6A shows that negatively charged PLG microparticles adhered to inflamed murine colonic tissue. Three (3) days after disease induction with TNBS, colons were resected from mice. Alternatively, colons were resected from age and sex matched, untreated mice. The colons were then cultured with fluorescently labelled microparticles. n=5 μmice per group. FIG. 6B shows the quantification of PLG microparticles adhered to inflamed murine colonic tissue. Statistical analysis was completed using one-way ANOVA with Tukey corrected for multiple comparisons.

FIGS. 7A-7C show degradation of factors in the gastric milieu without microparticle encapsulation. Microparticle encapsulation prevents degradation of bioactive factors in the gastric microenvironment. FIG. 7A shows the in silico prediction of cleavage of IL-2 protein (SEQ ID NO: 9) in the gastric environment with pepsin. FIG. 7B shows the in silico prediction of cleavage of TGF-β protein (SEQ ID NO: 1) in the gastric environment with pepsin. Underlined characters reflect predicted sites of protein cleavage. FIG. 7C shows the results of in vitro assay used to examine the change in IL-2 activity for free or microparticle encapsulated IL-2, in simulated gastric fluid with or without pepsin. The activity for the microparticle groups (MP) only accounted for the remaining activity within the microparticle, not the quantity of IL-2 that had been released. Statistics were completed using a student's t-test.

FIG. 8A-8F show the next generation colonic delivery system. Colonic delivery of microparticles can be enhanced by embedding microparticles within pH responsive hydrogels. FIG. 8A shows the chemical structure of the hydrogel components alginate and chitosan. FIG. 8B shows an illustration depicting ionic crosslinking of hydrogels. FIG. 8C shows an image captured after the formation of an ionically crosslinked hydrogel. FIG. 8D shows an image of hydrogel within tissue after administered orally. FIG. 8E shows microparticles can be embedded within the polymer. FIG. 8F shows microparticles embedded withing the polymer delayed the release of a model protein, bovine serum albumin, until the pH of the duodenum is achieved.

### DETAILED DESCRIPTION

The present disclosure relates to composition and methods useful in connection with use of microparticles to prevent and/or treat inflammatory bowel disease (IBD).

The inventors of the present disclosure discovered that the microparticles disclosed herein protect certain therapeutic agents (as disclosed in Section 2 below) from the pH-degradation occurring in the stomach pH and, and that the administration of these microparticles can reduce and revert the inflammation occurring in IBD.

For purposes of clarity of disclosure and not by way of limitation, the detailed description is divided into the following subsections:
1. Definitions;
2. Microparticles;
3. Pharmaceutical Compositions;
4. Methods of Treatment; and
5. Kits.

### 1. DEFINITIONS

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the disclosure and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.”

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of”, and “consisting essentially of,” the embodiments or elements presented herein, whether explicitly set forth or not.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing inflammatory bowel disease, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, and as well-understood in the art, “treatment” is an approach for obtaining beneficial or desired results, including clinical results. For purposes of the present disclosure, beneficial or desired clinical results include, but are not limited to, alleviation or amelioration of one or more sign or symptoms, diminishment of extent of disease, stabilized (i.e., not worsening) state of disease, prevention of disease, delay or slowing of disease progression, and/or amelioration or palliation of the disease state. The decrease can be a 10%, 20%, 30%, 40%, 50%, 60%, 70%, 80%, 90%, 95%, 98% or 99% decrease in severity of complications or symptoms. “Treatment” can also mean prolonging survival as compared to expected survival if not receiving treatment.

An “effective amount” or “therapeutically effective amount” is an amount effective, at dosages and for periods of time necessary, that produces a desired effect, e.g., the desired therapeutic or prophylactic result. In certain embodiments, an effective amount can be formulated and/or administered in a single dose. In certain embodiments, an effective amount can be formulated and/or administered in a plurality of doses, for example, as part of a dosing regimen.

The terms “inhibiting,” “reducing” or “prevention,” or any variation of these terms, referred to herein, includes any measurable decrease or complete inhibition to achieve a desired result. The benefit to a subject to be treated is either statistically significant or at least perceptible to the patient or to the physician. Treatment includes partial or full resolution of symptoms associated with the medical condition to be treated.

The terms “increasing” or “inducing,” or any variation of these terms, referred to herein, includes any measurable increase or complete activation to achieve a desired result. The benefit to a subject to be treated is either statistically significant or at least perceptible to the patient or to the physician. Treatment includes partial or full resolution of symptoms associated with the medical condition to be treated.

As used herein, the term “derivative” refers to a chemical compound with a similar core structure. For example, trichloromethane (chloroform) is a derivative of methane.

The term “enantiomers” refers to a pair of stereoisomers that are non-superimposable mirror images of each other. A 1:1 μmixture of a pair of enantiomers is a “racemic” mixture or a racemate. The term is used to designate a racemic mixture where appropriate.

The term “enantiopure” refers to a sample that within the limits of detection consists of a single enantiomer.

The term “diastereoisomers” refers to stereoisomers that have at least two asymmetric atoms, but which are not mirror-images of each other. The absolute stereochemistry is specified according to the Cahn-Ingold-Prelog R—S system. When a compound is a pure enantiomer, the stereochemistry at each chiral carbon can be specified by either R or S. Resolved compounds whose absolute configuration is unknown can be designated (+) or (−) depending on the direction (dextro or levorotatory) in which they rotate plane polarized light at the wavelength of the sodium D line.

The term “isomers” refers to different compounds that have the same molecular formula but differ in arrangement and configuration of the atoms. Also, as used herein, the term “stereoisomer” refers to any of the various stereo isomeric configurations which can exist for a given compound of the presently disclosed subject matter and includes geometric isomers. It is understood that a substituent can be attached at a chiral center of a carbon atom. Also, as used herein, the terms “constitutional isomers” refers to different compounds that have the same numbers of, and types of, atoms but the atoms are connected differently.

The term “nucleic acid molecule” and “nucleotide sequence,” as used herein, refers to a single or double-stranded covalently-linked sequence of nucleotides in which the 3′ and 5′ ends on each nucleotide are joined by phosphodiester bonds. The nucleic acid molecule can include deoxyribonucleotide bases or ribonucleotide bases, and can be manufactured synthetically in vitro or isolated from natural sources.

The terms “polypeptide,” “peptide,” “amino acid sequence” and “protein,” used interchangeably herein, refer to a molecule formed from the linking of at least two amino acids. The link between one amino acid residue and the next is an amide bond and is sometimes referred to as a peptide bond. A polypeptide can be obtained by a suitable method known in the art, including isolation from natural sources, expression in a recombinant expression system, chemical synthesis or enzymatic synthesis. The terms can apply to amino acid polymers in which one or more amino acid residue is an artificial chemical mimetic of a corresponding naturally occurring amino acid, as well as to naturally occurring amino acid polymers and non-naturally occurring amino acid polymers.

As used herein, “a functional fragment” of a molecule or polypeptide includes a fragment of the molecule or polypeptide that retains at least about 80%, at least about 85%, at least about 90%, at least about 95%, or at least about 100% of the primary function of the molecule or polypeptide.

### 2. Microparticles

The present disclosure provides microparticles for use in the methods disclosed herein. In certain embodiments, a microparticle can include a molecule, e.g., therapeutic agent, that inhibits the process of inflammation. In certain embodiments, a microparticle can include a molecule, e.g., therapeutic agent, that recruits regulatory T cells (Treg). In certain embodiments, a microparticle disclosed herein can reversibly or irreversibly inhibit the process involved in inflammatory bowel disease (e.g., resulting in decreased inflammation).

As used herein, a “microparticle” refers to any particle having a diameter of less than 1000 μm, e.g., from about 10 μm to about 200 μm. In certain embodiments, the microparticles can have a diameter of from about 10 μm to about 90 μm, from about 20 μm to about 80 μm, from about 60 μm to about 120 μm, from about 70 μm to about 120 μm, from about 80 am to about 120 μm, from about 90 μm to about 120 μm, from about 100 μm to about 120 μm, from about 60 μm to about 130 μm, from about 70 μm to about 130 μm, from about 80 am to about 130 μm, from about 90 μm to about 130 μm, from about 100 μm to about 130 μm, from about 110 μm to about 130 μm, from about 60 μm to about 140 μm, from about 70 am to about 140 μm, from about 80 μm to about 140 μm, from about 90 μm to about 140 μm, from about 100 μm to about 140 μm, from about 110 μm to about 140 μm, from about 60 μm to about 150 am, from about 70 μm to about 150 μm, from about 80 am to about 150 μm, from about 90 am to about 150 μm, from about 100 μm to about 150 μm, from about 110 μm to about 150 μm, or from about 120 μm to about 150 μm. In certain embodiments, the microparticles can have a diameter of from about 1 μm to about 30 μm, from about 2 μm to about 30 μm, from about 5 μm to about 30 μm, from about 7 μm to about 30 μm, from about 10 μm to about 30 μm, from about 12 μm to about 30 μm, from about 15 μm to about 30 μm, from about 20 μm to about 30 μm, from about 5 μm to about 20 μm, from about 8 μm to about 20 am, from about 10 μm to about 20 μm, from about 12 μm to about 20 μm, from about 15 am to about 20 μm, or from about 10 μm to about 15 μm. In certain embodiments, the microparticles can have a diameter of from about 10 μm to about 20 μm.

In certain embodiments, the microparticles can have a diameter of from about 10 nm to about 1000 nm, from about 50 nm to about 1000 nm, from about 100 nm to about 1000 nm, from about 150 nm to about 1000 nm, from about 200 nm to about 1000 nm, from about 300 nm to about 1000 nm, from about 400 nm to about 1000 nm, from about 500 nm to about 1000 nm, from about 600 nm to about 1000 nm, from about 700 nm to about 1000 nm, from about 800 nm to about 1000 nm, from about 100 nm to about 500 nm, from about 150 nm to about 500 nm, from about 200 nm to about 500 nm, from about 250 nm to about 500 nm, from about 300 nm to about 500 nm, from about 400 nm to about 500 nm, from about 500 nm to about 900 nm, from about 600 nm to about 900 nm, from about 700 nm to about 900 nm, from about 800 nm to about 900 nm, from about 100 nm to about 200 nm, from about 100 nm to about 300 nm, from about 100 nm to about 400 nm, from about 600 nm to about 800 nm, or from about 700 nm to about 800 nm. In certain embodiments, the microparticles can have a diameter of from about 10 nm to about 100 nm, from about 20 nm to about 100 nm, from about 50 nm to about 100 nm, from about 70 nm to about 100 nm, from about 110 nm to about 200 nm, from about 120 nm to about 100 nm, from about 150 nm to about 200 nm, from about 200 nm to about 300 nm, or from about 250 nm to about 300 nm.

In certain embodiments, the microparticle can include one or more lipids. In certain embodiments, the lipids can be neutral, anionic or cationic at physiological pH. In certain embodiments, the lipids can be sterols. For example, in certain embodiments, the lipid microparticle can include cholesterol, phospholipids and sphingolipids. In certain embodiments, the lipid microparticle can include steroids. Non-limiting examples of steroids include dexamethasone, methylprednisolone, prednisone, and prednisolone. In certain embodiments, the microparticles comprise PEGylated derivatives of the neutral, anionic, and cationic lipids. The incorporation of PEGylated derivatives can improve the stability of the microparticles. Non-limiting examples of PEGylated lipids include distearoylphosphatidylethanlamine- polyethylene glycol (DSPE-PEG), stearyl-polyethylene glycol and cholesteryl-polyethylene glycol. In certain embodiments, the microparticle include substituted or unsubstituted fatty acids. Non-limiting examples of saturated fatty acids include caproic acid, enanthic acid, caprylic acid, pelargonic acid, capric acid, undecanoic acid, lauric acid, tridecanoic acid, myristic acid, pentadecanoic acid, palmitic acid, margaric acid, stearic acid, nonadecanoic acid, arachidic acid, heneicosanoic acid, behenic acid, tricosanoic acid, lignoceric acid, pentacosanoic acid, cerotic acid, heptacosanoic acid, montanic acid, nonacosanoic acid, melissic acid, henatriacontanoic acid, lacceroic acid, psyllic acid, geddic acid, ceroplastic acid, hexatriacontanoic acid, and combinations thereof. Non-limiting examples of unsaturated fatty acids include hexadecatrienoic acid, alpha-linolenic acid, stearidonic acid, eicosatrienoic acid, eicosatetraenoic acid, eicosapentaenoic acid, heneicosapentaenoic acid, docosapentaenoic acid, docosahexaenoic acid, tetracosapentaenoic acid, tetracosahexaenoic acid, linoleic acid, gamma-linolenic acid, eicosadienoic acid, dihomo-gamma-linolenic acid, arachidonic acid, docosadienoic acid, adrenic acid, docosapentaenoic acid, tetracosatetraenoic acid, tetracosapentaenoic acid, oleic acid, eicosenoic acid, mead acid, erucic acid, nervonic acid, rumenic acid, α-calendic acid, β-calendic acid, jacaric acid, α-eleostearic acid, β-eleostearic acid, catalpic acid, punicic acid, rumelenic acid, α-parinaric acid, β-parinaric acid, bosseopentaenoic acid, pinolenic acid, podocarpic acid, palmitoleic acid, vaccenic acid, gadoleic acid, erucic acid, and combinations thereof.

In certain embodiments, the microparticles include polymers. In certain embodiments, the polymer can be amphiphilic, hydrophilic, or hydrophobic. In certain embodiments, the polymer can be biocompatible, e.g., the polymer does not induce an adverse and/or inflammatory response when administered to a subject. For example, without limitation, a polymer can be selected from polydioxanone (PDO), polyhydroxyalkanoate, polyhydroxybutyrate, poly(glycerol sebacate), polyglycolide (i.e., poly(glycolic) acid) (PGA), polylactide (i.e., poly(lactic) acid) (PLA), poly(lactic) acid-co-poly(glycolic) acid (PLGA), poly(lactide-co-glycolide) (PLG), polycaprolactone, copolymers, or derivatives including these and/or other polymers. In certain embodiments, the polymer includes PEG. In certain embodiments, the polymer includes poly(lactide-co-glycolide) (PLG).

In certain embodiments, the microparticles include cationic polymers. In certain embodiments, the cationic polymers can be branched or linear. Cationic polymers are able to condense and protect negatively charged molecules such as DNA or RNA. In certain embodiments, without limitation, the cationic polymers can be polyethylenimines, poly-histidyl polymers, chitosan, poly(amino ester glycol urethane), polylysines, or amino cyclodextrin derivatives. In certain embodiments, the microparticle comprises linear polyethylenimine. In certain embodiments, the microparticle comprises chitosan.

In certain embodiments, the microparticles include anionic polymers. In certain embodiments, the anionic polymers can be branched or linear. Anionic polymers are able to condense and protect positively charged molecules such as metals (e.g., Ca++) and positively charged proteins. In certain embodiments, without limitation, the anionic polymers can be polyacrylic acid cystamine conjugates and derivatives thereof, sodium carboxy methyl starch (CMS) and derivatives thereof, carboxy methyl guar gum (CMG) and derivatives thereof, carboxymethyl cellulose and derivatives thereof, or alginate and derivative thereof. In certain embodiments, the microparticle comprises alginate or a derivative thereof.

In certain embodiments, the microparticle can show organ tropism and can have an organ-specific distribution. In certain embodiments, the microparticles include molecules providing for organ tropism or organ-specific distribution. For example, but without any limitation, the surface of the microparticles can be functionalized to bind biological molecules (e.g., a ligand or an antibody) targeting a specific tissue (e.g., epithelial cells). The surface functionalization of microparticles can be based on the use of homo- or hetero-bifunctional cross linkers to the aim to add an organic functional group (e.g., R—NH2, R—COOH, etc.), useful to bind biological molecules (e.g., a ligand or an antibody). In certain embodiments, the functionalization of the surface of the microparticles can be achieved using non-covalent conjugation. In certain embodiments, the functionalization of the surface of the microparticles can be achieved using non-covalent conjugation. The covalent conjugation allows modifications at several levels using sequential functionalization and can be exploited to achieve structures with multiple functions. For example, without any limitation, the microparticle can include a PEG molecule synthesized with specific functional groups at the ends which can be used as homo-bifunctional or hetero-bifunctional linkers to perform a wide range of functionalization processes. In certain embodiments, the biological molecule is an antibody targeting an epithelial cell surface molecule. Non-limiting examples of epithelial cell surface molecules include A33, ACE/CD143, ALCAM/CD166, Aminopeptidase B/RNPEP, Aminopeptidase Inhibitors, Aminopeptidase N/CD13, Amnionless, B7-H2, B7-H3, CA125/MUC16, CA15-3/MUC-1, E-Cadherin, CD1a, CDld, CD1dl, CD46, CD74, CEACAM-1/CD66a, CEACAM-3/CD66d, CEACAM-4, CEACAM-5/CD66e, CEACAM-6/CD66c, CEACAM-7, Collagen I, CTRP5/C1qTNF5, Cubilin, DDR1, DDR1/DDR2, beta-Defensin 2, beta-Defensin 3, alpha-Defensin 1, alpha-Defensin 5, Endorepellin/Perlecan, EpCAM/TROP1, Fas Ligand/TNFSF6, Gastrokine 1, HIN-1/SCGB3A1, Hyaluronan, IGSF4C/SynCAM4, Integrin alpha 4/CD49d, Integrin alpha 4 beta 1, Integrin alpha 4 beta 7/LPAM-1, JAM-A, JAM-B/VE-JAM, JAM-C, L1CAM, Laminin-1, MFG-E8, MSPR/Ron, MUC-1, MUC-19, MUC-4, Nectin-1, Nectin-2/CD112, Nectin-3, Nectin-4, Nidogen-1/Entactin, Occludin, PD-L1/B7-H1, PLET-1, P1GF, Prostasin/Prss8, SLURP2, TfR (Transferrin R), and UGRP1/SCGB3A2.

In certain embodiments, the microparticle can adhere to specific tissues. In certain embodiments, the microparticle can adhere to a tissue of the digestive system. The digestive system includes an alimentary tract and accessory organs. The alimentary tract of the digestive system is composed of the mouth, pharynx, esophagus, stomach, small and large intestines, rectum, and anus. The accessory organs include salivary glands, liver, gallbladder, and pancreas.

In certain embodiments, the microparticles can adhere to a tissue (e.g., epithelium) of the mouth. In certain embodiments, the microparticles can adhere to a tissue (e.g., epithelium) of the pharynx. In certain embodiments, the microparticles can adhere to a tissue (e.g., epithelium) of the of the esophagus. In certain embodiments, the microparticles can adhere to a tissue (e.g., epithelium) of the stomach. In certain embodiments, the microparticles can adhere to a tissue (e.g., epithelium) of the small intestine. In certain embodiments, the microparticles can adhere to a tissue (e.g., epithelium) of the large intestine.

In certain embodiments, the microparticle can adhere to intestinal tissues. In certain embodiments, the microparticle can adhere to duodenum tissues. In certain embodiments, the microparticle can adhere to jejunum tissues. In certain embodiments, the microparticle can adhere to ileum tissues. In certain embodiments, the microparticle can adhere to colon tissue.

In certain embodiments, the microparticle can adhere to rectum tissues. In certain embodiments, the microparticle can adhere to anal canal tissues.

In certain embodiments, the microparticle can adhere to colon tissue. In certain embodiments, the colon tissue can be an ascending colon tissue, a transverse colon tissue, a descending colon tissue, or a sigmoid colon tissue. In certain embodiments, the microparticle can show cell tropism by binding of the ligand to a specific molecule on the cell. In certain embodiments, the cell can be an epithelial cell. In certain embodiments, the cell can be an intestinal cell. In certain embodiments, the cell can be an epithelial intestinal cell.

In certain embodiments, the microparticles can be biodegradable or non-biodegradable. In certain embodiment, the microparticle can be comprised in a pharmaceutical composition. Details on the pharmaceutical compositions contemplated by the present disclosure can be found in Section 3.

In certain embodiments, the microparticles include a therapeutic agent. Non-limiting examples of therapeutic agents encompassed by the present disclosure include all-trans retinoic acid, phorbol myristate acetate, indole-3-aldehyde, eupalitin-3-O-β-d-galactopyranoside, estrogen, kynurenine, butyrate, oleraceine, dexamethasone, methylprednisolone, prednisone, prendisolone, insulin-like growth factor-1, thymic stromal lymphopoietin, IL-15, and IL-7.

In certain embodiments, the therapeutic agent is a polypeptide. In certain embodiments, the therapeutic agent is a cytokine. In certain embodiments, the cytokine is a transforming growth factor beta (TGF-β) polypeptide or a functional fragment thereof. Transforming growth factor beta (TGF-β) is a multifunctional cytokine belonging to the transforming growth factor superfamily that includes three different mammalian isoforms (TGF-β-1, TGF-β-2, and TGF-β-3) and many other signaling proteins. TGF-β is involved in immune and stem cell regulation and differentiation. TGF-β has immunosuppressive functions and its dysregulation is implicated in the pathogenesis of autoimmune diseases.

In certain embodiment, the TGF-β polypeptide or functional fragment thereof is a human TGF-β polypeptide. In certain embodiments, the TGF-β polypeptide or functional fragment thereof comprises an amino acid sequence that is at least about 80% identical, 85% identical, 90% identical, 95% identical, 98% identical, or 99% identical to the amino acid sequence set forth in SEQ ID NO: 1. In certain embodiments, the TGF-β polypeptide comprises the amino acid sequence set forth in SEQ ID NO: 1. In certain embodiments, the TGF-β polypeptide or functional fragment thereof consists of an amino acid sequence that is at least about 80% identical, 85% identical, 90% identical, 95% identical, 98% identical, or 99% identical to the amino acid sequence set forth in SEQ ID NO: 1. In certain embodiments, the TGF-β polypeptide consists of the amino acid sequence set forth in SEQ ID NO: 1. SEQ ID NO: 1 is provided below.

(SEQ ID NO: 1)
MPPSGLRLLLLLLPLLWLLVLTPGRPAAGLSTCKTIDMELVKRKRIEA
IRGQILSKLRLASPPSQGEVPPGPLPEAVLALYNSTRDRVAGESAEPE
PEPEADYYAKEVTRVLMVETHNEIYDKFKQSTHSIYMFFNTSELREAV
PEPVLLSRAELRLLRLKLKVEQHVELYQKYSNNSWRYLSNRLLAPSDS
PEWLSFDVTGVVRQWLSRGGEIEGFRLSAHCSCDSRDNTLQVDINGFT
TGRRGDLATIHGMNRPFLLLMATPLERAQHLQSSRHRRALDTNYCFSS
TEKNCCVRQLYIDFRKDLGWKWIHEPKGYHANFCLGPCPYIWSLDTQY
SKVLALYNQHNPGASAAPCCVPQALEPLPIVYYVGRKPKVEQLSNMIV
RSCKCS

In certain embodiments, the TGF-β polypeptide or functional fragment thereof comprises an amino acid sequence that is at least about 80% identical, 85% identical, 90% identical, 95% identical, 98% identical, or 99% identical to the amino acid sequence set forth in SEQ ID NO: 2. In certain embodiments, the TGF-β polypeptide comprises the amino acid sequence set forth in SEQ ID NO: 2. In certain embodiments, the TGF-β polypeptide or functional fragment thereof consists of an amino acid sequence that is at least about 80% identical, 85% identical, 90% identical, 95% identical, 98% identical, or 99% identical to the amino acid sequence set forth in SEQ ID NO: 2. In certain embodiments, the TGF-β polypeptide consists of the amino acid sequence set forth in SEQ ID NO: 2. In certain embodiment, the TGF-β polypeptide or functional fragment thereof comprises two copies of the amino acid sequence set forth in SEQ ID NO: 2. In certain embodiments, the two copies of the amino acid sequence set forth in SEQ ID NO: 2 are linked by a disulfide bond. SEQ ID NO: 2 is provided below.

(SEQ ID NO: 2)
ALDTNYCFSSTEKNCCVRQLYIDFRKDLGWKWIHEPKGYHANFCLGPC
PYIWSLDTQYSKVLALYNQHNPGASAAPCCVPQALEPLPIVYYVGRKP
KVEQLSNMIVRSCKCS

In certain embodiments, the TGF-β polypeptide or functional fragment thereof comprises an amino acid sequence that is at least about 80% identical, 85% identical, 90% identical, 95% identical, 98% identical, or 99% identical to the amino acid sequence set forth in SEQ ID NO: 3. In certain embodiments, the TGF-β polypeptide comprises the amino acid sequence set forth in SEQ ID NO: 3. In certain embodiments, the TGF-β polypeptide or functional fragment thereof consists of an amino acid sequence that is at least about 80% identical, 85% identical, 90% identical, 95% identical, 98% identical, or 99% identical to the amino acid sequence set forth in SEQ ID NO: 3. In certain embodiments, the TGF-β polypeptide consists of the amino acid sequence set forth in SEQ ID NO: 3. SEQ ID NO: 3 is provided below.

(SEQ ID NO: 3)
MHYCVLSAFLILHLVTVALSLSTCSTLDMDQFMRKRIEAIRGQILSKL
KLTSPPEDYPEPEEVPPEVISIYNSTRDLLQEKASRRAAACERERSDE
EYYAKEVYKIDMPPFFPSENAIPPTFYRPYFRIVRFDVSAMEKNASNL
VKAEFRVERLQNPKARVPEQRIELYQILKSKDLTSPTQRYIDSKVVKT
RAEGEWLSFDVTDAVHEWLHHKDRNLGFKISLHCPCCTFVPSNNYIIP
NKSEELEARFAGIDGTSTYTSGDQKTIKSTRKKNSGKTPHLLLMLLPS
YRLESQQTNRRKKRALDAAYCFRNVQDNCCLRPLYIDFKRDLGWKWIH
EPKGYNANFCAGACPYLWSSDTQHSRVLSLYNTINPEASASPCCVSQD
LEPLTILYYIGKTPKIEQLSNMIVKSCKCS

In certain embodiments, the TGF-β polypeptide or functional fragment thereof comprises an amino acid sequence that is at least about 80% identical, 85% identical, 90% identical, 95% identical, 98% identical, or 99% identical to the amino acid sequence set forth in SEQ ID NO: 4. In certain embodiments, the TGF-β polypeptide comprises the amino acid sequence set forth in SEQ ID NO: 4. In certain embodiments, the TGF-β polypeptide or functional fragment thereof consists of an amino acid sequence that is at least about 80% identical, 85% identical, 90% identical, 95% identical, 98% identical, or 99% identical to the amino acid sequence set forth in SEQ ID NO: 4. In certain embodiments, the TGF-β polypeptide consists of the amino acid sequence set forth in SEQ ID NO: 4. In certain embodiment, the TGF-β polypeptide or functional fragment thereof comprises two copies of the amino acid sequence set forth in SEQ ID NO: 4. In certain embodiments, the two copies of the amino acid sequence set forth in SEQ ID NO: 4 are linked by a disulfide bond. SEQ ID NO: 4 is provided below.

(SEQ ID NO: 4)
ALDAAYCFRNVQDNCCLRPLYIDFKRDLGWKWIHEPKGYNANFCAGAC
PYLWSSDTQHSRVLSLYNTINPEASASPCCVSQDLEPLTILYYIGKTP
KIEQLSNMIVKSCKCS

In certain embodiments, the TGF-β polypeptide or functional fragment thereof comprises an amino acid sequence that is at least about 80% identical, 85% identical, 90% identical, 95% identical, 98% identical, or 99% identical to the amino acid sequence set forth in SEQ ID NO: 5. In certain embodiments, the TGF-β polypeptide comprises the amino acid sequence set forth in SEQ ID NO: 5. In certain embodiments, the TGF-β polypeptide or functional fragment thereof consists of an amino acid sequence that is at least about 80% identical, 85% identical, 90% identical, 95% identical, 98% identical, or 99% identical to the amino acid sequence set forth in SEQ ID NO: 5. In certain embodiments, the TGF-β polypeptide consists of the amino acid sequence set forth in SEQ ID NO: 5. SEQ ID NO: 5 is provided below.

(SEQ ID NO: 5)
MKMHLQRALVVLALLNFATVSLSLSTCTTLDFGHIKKKRVEAIRGQIL
SKLRLTSPPEPTVMTHVPYQVLALYNSTRELLEEMHGEREEGCTQENT
ESEYYAKEIHKFDMIQGLAEHNELAVCPKGITSKVFRFNVSSVEKNRT
NLFRAEFRVLRVPNPSSKRNEQRIELFQILRPDEHIAKQRYIGGKNLP
TRGTAEWLSFDVTDTVREWLLRRESNLGLEISIHCPCHTFQPNGDILE
NIHEVMEIKFKGVDNEDDHGRGDLGRLKKQKDHHNPHLILMMIPPHRL
DNPGQGGQRKKRALDTNYCFRNLEENCCVRPLYIDFRQDLGWKWVHEP
KGYYANFCSGPCPYLRSADTTHSTVLGLYNTLNPEASASPCCVPQDLE
PLTILYYVGRTPKVEQLSNMVVKSCKCS

In certain embodiments, the TGF-β polypeptide or functional fragment thereof comprises an amino acid sequence that is at least about 80% identical, 85% identical, 90% identical, 95% identical, 98% identical, or 99% identical to the amino acid sequence set forth in SEQ ID NO: 6. In certain embodiments, the TGF-β polypeptide comprises the amino acid sequence set forth in SEQ ID NO: 6. In certain embodiments, the TGF-β polypeptide or functional fragment thereof consists of an amino acid sequence that is at least about 80% identical, 85% identical, 90% identical, 95% identical, 98% identical, or 99% identical to the amino acid sequence set forth in SEQ ID NO: 6. In certain embodiments, the TGF-β polypeptide consists of the amino acid sequence set forth in SEQ ID NO: 6. In certain embodiment, the TGF-β polypeptide or functional fragment thereof comprises two copies of the amino acid sequence set forth in SEQ ID NO: 6. In certain embodiments, the two copies of the amino acid sequence set forth in SEQ ID NO: 6 are linked by a disulfide bond. SEQ ID NO: 6 is provided below.

(SEQ ID NO: 6)
ALDTNYCFRNLEENCCVRPLYIDFRQDLGWKWVHEPKGYYANFCSGPC
PYLRSADTTHSTVLGLYNTLNPEASASPCCVPQDLEPLTILYYVGRTP
KVEQLSNMVVKSCKCS

In certain embodiments, the cytokine is an interleukin. In certain embodiments, the interleukin is an Interleukin-2 (IL-2) polypeptide or a functional fragment thereof. Interleukin-2 (IL-2) is a type of cytokine signaling molecule in the immune system that regulates the activities of lymphocytes that are responsible for immunity. IL-2 has essential roles in key functions of the immune system, tolerance, and immunity, primarily via its direct effects on T cells. While IL-2 is generally considered to promote T-cell proliferation and enhance effector T-cell function, it has been demonstrated that treatments that utilize low-dose IL-2 unexpectedly induce immune tolerance and promote Treg development resulting in the suppression of unwanted immune responses and eventually leading to treatment of some autoimmune disorders.

In certain embodiment, the IL-2 polypeptide or functional fragment thereof is a murine IL-2 polypeptide. In certain embodiments, the IL-2 polypeptide or functional fragment thereof comprises an amino acid sequence that is at least about 80% identical, 85% identical, 90% identical, 95% identical, 98% identical, or 99% identical to the amino acid sequence set forth in SEQ ID NO: 7. In certain embodiments, the IL-2 polypeptide comprises the amino acid sequence set forth in SEQ ID NO: 7. In certain embodiments, the IL-2 polypeptide or functional fragment thereof consists of an amino acid sequence that is at least about 80% identical, 85% identical, 90% identical, 95% identical, 98% identical, or 99% identical to the amino acid sequence set forth in SEQ ID NO: 7. In certain embodiments, the IL-2 polypeptide consists of the amino acid sequence set forth in SEQ ID NO: 7. SEQ ID NO: 7 is provided below.

(SEQ ID NO: 7)
MYSMQLASCVTLTLVLLVNSAPTSSSTSSSTAEAQQQQQQQQQQQQHL
EQLLMDLQELLSRMENYRNLKLPRMLTFKFYLPKQATELKDLQCLEDE
LGPLRHVLDLTQSKSFQLEDAENFISNIRVTVVKLKGSDNTFECQFDD
ESATVVDFLRRWIAFCQSIISTSPQ

In certain embodiments, the IL-2 polypeptide or functional fragment thereof comprises an amino acid sequence that is at least about 80% identical, 85% identical, 90% identical, 95% identical, 98% identical, or 99% identical to the amino acid sequence set forth in SEQ ID NO: 8. In certain embodiments, the IL-2 polypeptide comprises the amino acid sequence set forth in SEQ ID NO: 8. In certain embodiments, the IL-2 polypeptide or functional fragment thereof consists of an amino acid sequence that is at least about 80% identical, 85% identical, 90% identical, 95% identical, 98% identical, or 99% identical to the amino acid sequence set forth in SEQ ID NO: 8. In certain embodiments, the IL-2 polypeptide consists of the amino acid sequence set forth in SEQ ID NO: 8. SEQ ID NO: 8 is provided below.

(SEQ ID NO: 8)
APTSSSTSSSTAEAQQQQQQQQQQQQHLEQLLMDLQELLSRMENYRNL
KLPRMLTFKFYLPKQATELKDLQCLEDELGPLRHVLDLTQSKSFQLED
AENFISNIRVTVVKLKGSDNTFECQFDDESATVVDFLRRWIAFCQSII
STSPQ

In certain embodiment, the IL-2 polypeptide or functional fragment thereof is a human IL-2 polypeptide. In certain embodiments, the IL-2 polypeptide or functional fragment thereof comprises an amino acid sequence that is at least about 80% identical, 85% identical, 90% identical, 95% identical, 98% identical, or 99% identical to the amino acid sequence set forth in SEQ ID NO: 9. In certain embodiments, the IL-2 polypeptide comprises the amino acid sequence set forth in SEQ ID NO: 9. In certain embodiments, the IL-2 polypeptide or functional fragment thereof consists of an amino acid sequence that is at least about 80% identical, 85% identical, 90% identical, 95% identical, 98% identical, or 99% identical to the amino acid sequence set forth in SEQ ID NO: 9. In certain embodiments, the IL-2 polypeptide consists of the amino acid sequence set forth in SEQ ID NO: 9. SEQ ID NO: 9 is provided below.

(SEQ ID NO: 9)
MYRMQLLSCIALSLALVINSAPTSSSTKKTQLQLEHLLLDLQMILNGI
NNYKNPKLTRMLTFKFYMPKKATELKHLQCLEEELKPLEEVLNLAQSK
NFHLRPRDLISNINVIVLELKGSETTFMCEYADETATIVEFLNRWITF
CQSIISTLT

In certain embodiments, the IL-2 polypeptide or functional fragment thereof comprises an amino acid sequence that is at least about 80% identical, 85% identical, 90% identical, 95% identical, 98% identical, or 99% identical to the amino acid sequence set forth in SEQ ID NO: 10. In certain embodiments, the IL-2 polypeptide comprises the amino acid sequence set forth in SEQ ID NO: 10. In certain embodiments, the IL-2 polypeptide or functional fragment thereof consists of an amino acid sequence that is at least about 80% identical, 85% identical, 90% identical, 95% identical, 98% identical, or 99% identical to the amino acid sequence set forth in SEQ ID NO: 10. In certain embodiments, the IL-2 polypeptide consists of the amino acid sequence set forth in SEQ ID NO: 10. SEQ ID NO: 10 is provided below.

(SEQ ID NO: 10)
APTSSSTKKTQLQLEHLLLDLQMILNGINNYKNPKLTRMLTFKFYMPK
KATELKHLQCLEEELKPLEEVLNLAQSKNFHLRPRDLISNINVIVLEL
KGSETTFMCEYADETATIVEFLNRWITFCQSIISTLT

In certain embodiments, the therapeutic agent is a polypeptide including a conservative amino acid substitution. In certain embodiments, the TGF-β polypeptide or functional fragment thereof comprises a conservative amino acid substitution. In certain embodiments, the IL-2 polypeptide or functional fragment thereof comprises a conservative amino acid substitution.

In certain embodiments, conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. Amino acids can also be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. In certain embodiments, no more than one, no more than two, no more than three, no more than four, no more than five residues within a specified sequence are altered. Exemplary conservative amino acid substitutions are shown in Table 1 below.

TABLE 1

| Original Residue | Exemplary Conservative Amino Acid Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gin (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala |

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 μmatrix or a PAM250 matrix, and a gap weight of 16, 14, 12, 10, 8, 6, or 4 and a length weight of 1, 2, 3, 4, 5, or 6.

In certain embodiments, the therapeutic agent is an immunosuppressant. In certain embodiments, the immunosuppressant is an mTOR inhibitor. In certain embodiments, the immunosuppressant is a macrolide. Macrolides are a group of antibiotics with a distinctive macrocyclic lactone ring combined with sugars (e.g., cladinose, desosamine). The action of macrolides is to block protein synthesis by binding to the subunits of ribosome. Macrolides have also immunomodulatory effects since they inhibit the production of proinflammatory cytokines (e.g., TNF, IL-1, IL-6, and IL-8), affect transcription factors (e.g., NF-κB) as well as costimulaton (e.g., CD 80) and adhesion molecules (e.g., ICAM). Non-limiting examples of macrolides include azithromycin, clarithromycin, erythromycin, fidaxomicin, carbomycin A, josamycin, kitasamycin, midecamycin/midecamycin acetate, oleandomycin, solithromycin, spiramycin, troleandomycin, roxithromycin, telithromycin, cethromycin, tacrolimus, pimecrolimus, everolimus, and rapamycin. In certain embodiments, the macrolide is rapamycin or a derivative thereof. In certain embodiments, the rapamycin has the formula:

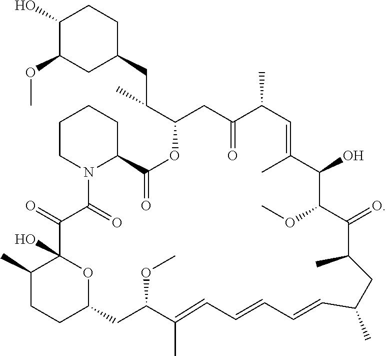 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

In certain embodiments, the macrolide is everolimus or a derivative thereof. In certain embodiments, the everolimus has the formula:

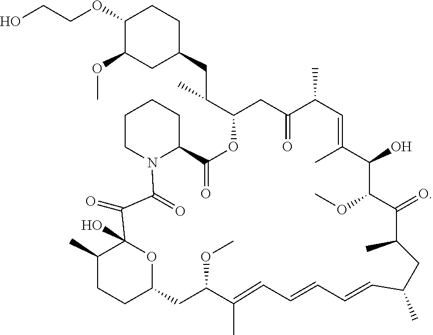 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C]

In certain embodiments, the microparticle includes stereoisomers, enantiomers, diastereomers, or racemates of the macrolide (e.g., rapamycin). The macrolides can contain one or more asymmetric centers and can thus give rise to enantiomers, diastereomers, and other stereoisomeric forms that can be defined, in terms of absolute stereochemistry, as (R)- or (S)—. In certain embodiments, the microparticle includes all possible isomers, including racemic mixtures, optically pure forms, and intermediate mixtures of the macrolide. Optically active (R)— and (S)— isomers can be prepared using chiral synthons or chiral reagents or resolved using conventional techniques. If the macrolide contains a double bond, the substituent can be E or Z configuration. If the macrolide contains a disubstituted cycloalkyl, the cycloalkyl substituent can have a cis- or trans-configuration. All tautomeric forms are also intended to be included.

In certain embodiments, the microparticle comprises a therapeutic agent (e.g., TGF-β, IL-2, rapamycin) and a polymer (e.g., PLG). In certain embodiments, the weight ratio of the therapeutic agent and the polymer can be between about 1:100000 and about 1:1, between about 1:100000 and about 1:2, between about 1:100000 and about 1:5, between about 1:100000 and about 1:10, between about 1:100000 and about 1:20, between about 1:50000 and about 1:1, between about 1:50000 and about 1:2, between about 1:50000 and about 1:5, between about 1:50000 and about 1:10, be between about 1:30000 and about 1:1, between about 1:30000 and about 1:2, between about 1:30000 and about 1:5, between about 1:30000 and about 1:10, between about 1:20000 and about 1:1, between about 1:20000 and about 1:2, between about 1:20000 and about 1:5, between about 1:10000 and about 1:1, between about 1:10000 and about 1:2, between about 1:10000 and about 1:5, between about 1:5000 and about 1:1, or between about 1:5000 and about 1:2. In certain embodiments, the weight ratio is between about 1:20000 and about 1:1.

In certain embodiments, the microparticle can provide a controlled release of the therapeutic agent. The term “controlled release” refers to any microparticle in which the manner and profile of a therapeutic agent release (e.g., TGF-β, IL-2, rapamycin) from the microparticle are controlled. This refers to immediate as well as non-immediate release microparticles, with non-immediate release microparticles including but not limited to sustained release and delayed release microparticles.

In certain embodiments, the microparticle can provide a sustained release of the therapeutic agent. The term “sustained release” refers to a microparticle that provides for gradual release of a therapeutic agent (e.g., TGF-β, IL-2, rapamycin) over an extended period of time. In certain embodiments, sustained release results in constant blood levels of a therapeutic agent over an extended time period.

In certain embodiments, the microparticle can provide a delayed release of the therapeutic agent. The term “delayed release” refers to a microparticle in which there is a time delay between administration of the microparticle and the release of the therapeutic agent. “Delayed release” can involve gradual release of a therapeutic agent over an extended period of time, and thus can be “sustained release.”

In certain embodiments, the microparticle can provide a long-term release of the therapeutic agent. Use of a long-term sustained release can be particularly useful for treatment of chronic conditions (e.g., inflammatory bowel disease). As used herein, “long-term release” refers to a microparticle capable of delivering therapeutic levels of the agent for at least 7 days, at least 15 days, at least 30 days, or at least 60 days.

Additional information on the features and components of the microparticles disclosed herein can be found in International Patent Publication Nos. WO2011006029, WO2013112456, and WO2014022685, the content of each of which is incorporated by reference in their entireties.

### 3. Pharmaceutical Formulation

In certain non-limiting embodiments, the present disclosure further provides pharmaceutical formulations of microparticles for therapeutic use. In certain embodiments, the pharmaceutical formulation includes a microparticle disclosed herein and a pharmaceutically acceptable carrier. “Pharmaceutically acceptable,” as used herein, includes any carrier which does not interfere with the effectiveness of the biological activity of the active ingredients, e.g., microparticles or therapeutic agent, and that is not toxic to the patient to whom it is administered. Non-limiting examples of suitable pharmaceutical carriers include phosphate-buffered saline solutions, water, emulsions, such as oil/water emulsions, various types of wetting agents and sterile solutions. Additional non-limiting examples of pharmaceutically acceptable carriers can include gels, bioabsorbable matrix materials, implantation elements containing the microparticles and/or any other suitable vehicle, delivery or dispensing means or material. Such carriers can be formulated by conventional methods and can be administered to the subject.

In certain embodiments, the pharmaceutical formulations of the present disclosure can be formulated using pharmaceutically acceptable carriers well known in the art that are suitable for parenteral administration, e.g., intravenous administration, intraarterial administration, intrathecal administration, intranasal administration, intramuscular administration, subcutaneous administration and intracisternal administration. In certain embodiments, the pharmaceutical formulation is formulated for intrathecal administration. For example, but not by way of limitation, the pharmaceutical formulation can be formulated as solutions, suspensions, or emulsions.

In certain non-limiting embodiments, the pharmaceutical formulations of the present disclosure can be formulated using pharmaceutically acceptable carriers well known in the art that are suitable for oral administration. Such carriers enable the pharmaceutical compositions to be formulated as tablets, pills, capsules, liquids, gels, syrups, slurries, suspensions and the like, for oral ingestion by a patient to be treated. In certain embodiments, the pharmaceutical formulation can be a solid dosage form.

In certain embodiments, the presently disclosed pharmaceutical formulations suitable for oral administration can be in the form of capsules (e.g., sprinkle capsules and gelatin capsules), cachets, pills, tablets, lozenges, lyophile, powders, granules, as a solution or a suspension in an aqueous or non-aqueous liquid, as an oil-in-water or water-in-oil liquid emulsion, as an elixir or syrup, and/or as pastilles. In certain embodiments, the pharmaceutical formulation contains a predetermined amount of the microparticles disclosed herein as an active ingredient.

In certain embodiments, the pharmaceutical formulation suitable for oral administration includes microparticles mixed with one or more pharmaceutically acceptable carriers (e.g., sodium citrate or dicalcium phosphate), fillers or extenders (e.g., starches, lactose, sucrose, glucose, mannitol, silicic acid), binders (e.g., carboxymethylcellulose, alginates, gelatin, polyvinyl pyrrolidone, sucrose, acacia), humectants (e.g., glycerol), disintegrating agents (e.g., calcium carbonate, alginic acid, certain silicates, sodium carbonate), solution retarding agents (e.g., paraffin), absorption accelerators (e.g., quaternary ammonium compounds), wetting agents (e.g., cetyl alcohol and glycerol monostearate), absorbents (e.g., kaolin and bentonite clay) lubricants (e.g., talc, calcium stearate, magnesium stearate, solid polyethylene glycols, sodium lauryl sulfate, and mixtures thereof), complexing agents (e.g., modified and unmodified cyclodextrins), coloring agents, or any combination thereof. In certain embodiments, the pharmaceutical formulation includes buffering agents.

In certain embodiments, the pharmaceutical formulation suitable for oral administration can include microparticles embedded in hydrogels. “Hydrogel” refers to a substance formed when an organic polymer (natural or synthetic) is cross-linked via covalent, ionic, or hydrogen bonds to create a three-dimensional open-lattice structure that entraps water molecules to form a gel. In certain embodiments, water molecules are the majority of the mass of the hydrogel.

In certain embodiments, the microparticles are encapsulated using an anionic polymer such as alginate to provide the hydrogel layer (e.g., core), where the hydrogel layer is subsequently cross-linked with a polycationic polymer (e.g., an amino acid polymer such as polylysine) to form a shell. In certain embodiments, the microparticles are embedded in hydrogels comprising alginate. Alginate forms a gel in the presence of divalent cations via ionic crosslinking. Notably, alginate does not degrade but dissolves when the divalent cations are replaced by monovalent ions.

In certain embodiments, the microparticles are embedded in hydrogels comprising chitosan. Chitosan is made by partially deacetylating chitin, a natural nonmammalian polysaccharide, which exhibits a close resemblance to mammalian polysaccharides, making it attractive for cell encapsulation. Under dilute acid conditions (pH<6), chitosan is positively charged and water-soluble, while at physiological pH, chitosan is neutral and hydrophobic, leading to the formation of a solid physically crosslinked hydrogel. In certain embodiments, the microparticles are embedded in hydrogels comprising alginate and chitosan.

In certain embodiments, suitable for oral administration can be a liquid dosage. In certain non-limiting embodiments, the liquid dosage includes pharmaceutically acceptable emulsions, microemulsions, solutions, suspensions, syrups, and elixirs. In certain embodiments, the liquid dosage contains inert diluents, water or other solvents, cyclodextrins and derivatives thereof, solubilizing agents and emulsifiers, ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols, fatty acid esters of sorbitan, and mixtures thereof.

Alternatively or additionally, the present disclosure provides pharmaceutical formulations for delivery via a catheter, stent, wire, or other intraluminal devices. Delivery via such devices can be especially useful for delivery to the rectum or intestine.

In certain embodiments, the pharmaceutical formulation can be formulated to release the microparticles immediately upon administration. Alternatively, the pharmaceutical formulation can be formulated to release the microparticles at any predetermined time or time period after administration. In certain embodiments, the pharmaceutical formulation can localize action of the microparticles, e.g., spatial placement of a controlled release adjacent to or in the disease, e.g., intestinal cells. In certain embodiments, the pharmaceutical formulation can achieve convenience of dosing, e.g., administering the formulation once per week or once every two weeks.

In certain embodiments, the pharmaceutical formulation can include from about 0.05 mg to about 100 mg of microparticles. In certain embodiments, the pharmaceutical formulation can include up to about 2,000 mg of the microparticles disclosed herein. For example, but not by way of limitation, the pharmaceutical formulation can include up to about 1,950 mg, up to about 1,900 mg, up to about 1,850 mg, up to about 1,800 mg, up to about 1,750 mg, up to about 1,700 mg, up to about 1,650 mg, up to about 1,600 mg, up to about 1,550 mg, up to about 1,500 mg, up to about 1,450 mg, up to about 1,400 mg, up to about 1,350 mg, up to about 1,300 mg, up to about 1,250 mg, up to about 1,200 mg, up to about 1,150 mg, up to about 1,100 mg, up to about 1,050 mg, up to about 1,000 mg, up to about 950 mg, up to about 900 mg, up to about 850 mg, up to about 800 mg, up to about 750 mg, up to about 700 mg, up to about 650 mg, up to about 600 mg, up to about 550 mg, up to about 500 mg, up to about 450 mg, up to about 400 mg, up to about 350 mg, up to about 300 mg, up to about 250 mg, up to about 200 mg, up to about 150 mg, up to about 100 mg, up to about 50 mg or up to about 25 mg of the microparticles disclosed herein. In certain embodiments, the pharmaceutical formulation can include up to about 25 mg of the microparticles disclosed herein. For example, but not by way of limitation, the pharmaceutical formulation can include up to about 1 mg, up to about 1.5 mg, up to about 2 mg, up to about 3 mg, up to about 5 mg, up to about 7 mg, up to about 8 mg, up to about 10 mg, up to about 12 mg, up to about 15 mg, up to about 18 mg, up to about 20 mg, up to about 22 mg, up to about 24 mg, or up to about 25 mg of the microparticles disclosed herein. In certain embodiments, the pharmaceutical formulation includes from about 1 mg to about 25 mg of the microparticles disclosed herein.

In certain embodiments, the pharmaceutical formulation disclosed herein includes a therapeutic agent at an amount of from about 0.0001 μg to about 10 μg per mg of microparticle. For example, but not by way of limitation, a pharmaceutical formulation includes up to about 9.5 μg, up to about 9 μg, up to about 8.5 μg, up to about 8 μg, up to about 7.5 μg, up to about 7 μg, up to about 6.5 μg, up to about 6 μg, up to about 5.5 μg, up to about 5 μg, up to about 4.5 g, up to about 4 μg, up to about 3.5 μg, up to about 3 μg, up to about 2.5 μg, up to about 2 μg, up to about 1.5 μg, up to about 1 μg, up to about 0.5 μg, up to about 0.1 μg, up to about 0.05 g, up to about 0.01 μg, up to about 0.005 μg, up to about 0.001 μg, up to about 0.0005 μg, or up to about 0.001 μg per mg of microparticle. In certain embodiments, the pharmaceutical formulation includes 1.7 μg per mg of microparticle. In certain embodiments, the pharmaceutical formulation includes 0.009 μg per mg of microparticle.

In certain embodiments, the pharmaceutical formulation disclosed herein includes a therapeutic agent at an amount of from about 10 μg to about 1000 μg per mg of microparticle. For example, but not by way of limitation, a pharmaceutical formulation includes up to about 950 μg, up to about 900 μg, up to about 850 μg, up to about 800 μg, up to about 750 μg, up to about 700 μg, up to about 650 μg, up to about 600 μg, up to about 550 μg, up to about 500 μg, up to about 450 μg, up to about 400 μg, up to about 350 μg, up to about 300 μg, up to about 250 μg, up to about 200 μg, up to about 150 μg, up to about 100 μg, up to about 50 μg, up to about 40 μg, up to about 30 μg, or up to about 20 μg per mg of microparticle.

### 3.1. Exemplary Pharmaceutical Formulation

In certain embodiments, the pharmaceutical formulation disclosed herein comprises a first microparticle, a second microparticle, and a third microparticle. In certain embodiments, each of the first microparticle, the second microparticle, and the third microparticle comprise a therapeutic agent (e.g., TGF-β, IL-2, rapamycin). In certain embodiments, the first microparticle, the second microparticle, and the third microparticle comprise different therapeutic agents. In certain embodiments, the first microparticle comprises a first therapeutic agent. In certain embodiments, the first therapeutic agent is TGF-β. In certain embodiments, the second microparticle comprises a second therapeutic agent. In certain embodiments, the second therapeutic agent is IL-2. In certain embodiments, the third microparticle comprises a third therapeutic agent. In certain embodiments, the third therapeutic agent is rapamycin. In certain embodiments, the pharmaceutical formulation is formulated for oral administration to a subject. In certain embodiments, the pharmaceutical formulation provides a therapeutically effective amount of a first therapeutic agent, a therapeutically effective amount of a second therapeutic agent, and a therapeutically effective amount of a third therapeutic agent. In certain embodiments, the pharmaceutical formulation is used for preventing and/or treating IBD in a subject. In certain embodiments, pharmaceutical formulation inhibits the development and growth of inflamed intestinal tissues in a subject.

In certain embodiments, the pharmaceutical formulation is formulated for oral administration to a subject. In certain embodiments, the pharmaceutical formulation provides a therapeutically effective amount of a first therapeutic agent, a therapeutically effective amount of a second therapeutic agent, and a therapeutically effective amount of a third therapeutic agent. In certain embodiments, the pharmaceutical formulation is used for preventing and/or treating a gastrointestinal condition in a subject. In certain embodiments, pharmaceutical formulation inhibits the development and growth of inflamed intestinal tissues in a subject. In certain embodiments, the gastrointestinal condition is selected from the group consisting of ulcerative colitis, Crohn's disease, gastritis, peptic ulcers, oesophagitis, cholecystitis, Gastro-Intestinal Graft Versus Host Disease (GI-GVHD), gastrointestinal cancers or tumors, gastrointestinal infections, and gastrointestinal immunopathies.

### 4. Methods of Treatment

The present disclosure relates to methods for preventing and/or treating inflammatory bowel disease (IBD) in a subject. The present disclosure provides methods for preventing and/or treating IBD in a subject by reducing local inflammation of the intestinal milieu. As described in detail in the Example section below, the studies presented in the instant application indicate that microparticles including anti-inflammatory therapeutic agents (as disclosed in Section 2) can be used to prevent and/or treat IBD by inhibiting the inflammation process.

Inflammatory bowel disease (IBD) can be mainly divided into ulcerative colitis (UC) and Crohn's disease (CD). Crohn's disease is similar to UC, both of which have been classified as chronic IBD and which cause digestive disorders and inflammation in the gastrointestinal tract. Some of the symptoms of CD and UC include diarrhea, abdominal pain, rectal bleeding, and weight loss. These diseases are mainly characterized by inflammation.

Crohn's disease is one of the IBDs that occur in patients between ages 15-35 years. Unlike other inflammatory diseases, IBDs could not be suppressed easily. Consequently, the immune system is stimulated, and part of the intestine is destroyed. It causes pain, diarrhea, fever, and other symptoms. In addition to the serious effect on the lower part of the small intestine, CD can also occur in parts of the digestive tract including the large intestine, stomach, esophagus, or even mouth. Crohn's disease affects the mouth, anus, and entire layers of the intestine while ulcerative colitis affects the mucosal layer of the colon. Ulcerative colitis is associated with blood in stool, severe pain, and diarrhea, while in CD there is also a risk of bleeding in severe cases. The affected areas of the digestive tract vary in these diseases. For example, CD often affects the ileum and a part of the large intestine. In CD, the small intestine often becomes inflamed, while UC is limited to the colon and is found mostly in some parts of the large intestine including colon and rectum. In UC, the large intestine becomes inflamed and the small intestine works naturally. Ulcerative colitis only affects the innermost part of the colon, while CD occurs in all layers of the bowel wall.

In certain non-limiting embodiments, the present disclosure provides for a method of preventing and/or treating inflammatory bowel disease in a subject. In certain embodiments, the present disclosure provides for a method of preventing and/or treating Crohn's disease in a subject. In certain embodiments, the present disclosure provides for a method of preventing and/or treating ulcerative colitis in a subject. In certain embodiments, the method can include administering a therapeutically effective amount of a microparticle disclosed herein to the subject. In certain embodiments, administration of the microparticle inhibits the development and growth of inflamed intestinal tissues in a subject. In certain embodiments, the subject was known to have inflammatory bowel disease prior to treatment. In certain non-limiting embodiments, the subject was not known to have inflammatory bowel disease prior to treatment.

In certain non-limiting embodiments, the present disclosure provides a method of treating a subject having inflammatory bowel disease (IBD) that includes diagnosing IBD in the subject and then treating the subject with a microparticle disclosed herein. In certain non-limiting embodiments, the method for diagnosing IBD includes determining levels of red blood cells, white blood cells, platelets, C-reactive protein, or infectious pathogens in the stools. Additional methods for diagnosing IBD include endoscopic procedures, computerized tomography (CT), and magnetic resonance imaging.

In certain non-limiting embodiments, the present disclosure provides for a method of preventing the development and/or growth of inflammation in a subject having IBD. In certain embodiments, the method includes administering a therapeutically effective amount of a microparticle disclosed herein to the subject. In certain embodiments, preventing inflammation includes recruiting in the endothelium of a subject.

Alternatively or additionally, the present disclosure provides for a method of preventing or treating a gastrointestinal condition. As used herein, the term “gastrointestinal condition” refers to a disease or disorder involving the gastrointestinal tract including, without any limitation, the esophagus, stomach, small intestine, large intestine and rectum, and the accessory organs of digestion, the liver, gallbladder, and pancreas. In certain embodiments, the gastrointestinal condition is associated with inflammation of the tissues of the digestive tract. In certain embodiments, the gastrointestinal condition is selected from the group of gastritis, peptic ulcers, esophagitis, cholecystitis, Gastro-Intestinal Graft Versus Host Disease (GI-GVHD), gastrointestinal cancers or tumors, gastrointestinal infections, and gastrointestinal immunopathies. In certain embodiments, gastrointestinal immunopathies can include autoimmune enteritis, eosinophil enteritis, neutrophilic colitis, check point inhibitor enteritis and colitis, microscopic colitis, and ischemic colitis.

In certain embodiments, a microparticle disclosed herein can be administered to a subject at a dose of about 0.05 mg/kg to about 500 mg/kg. In certain embodiments, a subject can be administered a dose up to about 500 mg/kg of the microparticle in a single dose or as a total daily dose. For example, but not by way of limitation, a subject can be administered up to about 450 mg/kg, up to about 400 mg/kg, up to about 350 mg/kg, up to about 300 mg/kg, up to about 250 mg/kg, up to about 200 mg/kg, up to about 150 mg/kg, up to about 100 mg/kg, up to about 50 mg/kg, up to about 25 mg/kg, up to about 20 mg/kg, up to about 15 mg/kg, up to about 10 mg/kg, up to about 5 mg/kg, up to about 4 mg/kg, up to about 2 mg/kg, up to about 1 mg/kg, up to about 0.5 mg/kg, up to about 0.1 mg/kg, or up to about 0.05 mg/kg.

In certain embodiments, a subject can be administered up to about 2,000 mg of the microparticle in a single dose or as a total daily dose. For example, but not by way of limitation, a subject can be administered up to about 1,950 mg, up to about 1,900 mg, up to about 1,850 mg, up to about 1,800 mg, up to about 1,750 mg, up to about 1,700 mg, up to about 1,650 mg, up to about 1,600 mg, up to about 1,550 mg, up to about 1,500 mg, up to about 1,450 mg, up to about 1,400 mg, up to about 1,350 mg, up to about 1,300 mg, up to about 1,250 mg, up to about 1,200 mg, up to about 1,150 mg, up to about 1,100 mg, up to about 1,050 mg, up to about 1,000 mg, up to about 950 mg, up to about 900 mg, up to about 850 mg, up to about 800 mg, up to about 750 mg, up to about 700 mg, up to about 650 mg, up to about 600 mg, up to about 550 mg, up to about 500 mg, up to about 450 mg, up to about 400 mg, up to about 350 mg, up to about 300 mg, up to about 250 mg, up to about 200 mg, up to about 150 mg, up to about 100 mg, up to about 50 mg or up to about 25 mg of the microparticle in a single dose or as a total daily dose. In certain embodiments, the subject can be administered from about 50 mg to about 1,000 mg of the microparticle in a single dose or a total daily dose. In certain embodiments, a subject can be administered about 1,000 mg of the microparticle in a single dose or as a total daily dose. In certain embodiments, a subject can be administered about 25 mg or more of the microparticles in a single dose or as a total daily dose.

In certain embodiments, the therapeutic agent can be released by the microparticle at a dose of about 0.05 ng/kg to about 100 ng/kg. In certain embodiments, a subject can be administered up to about 2,000 ng of the therapeutic agent released by the microparticle in a single dose or as a total daily dose. For example, but not by way of limitation, a subject can be administered up to about 1,950 ng, up to about 1,900 ng, up to about 1,850 ng, up to about 1,800 ng, up to about 1,750 ng, up to about 1,700 ng, up to about 1,650 ng, up to about 1,600 ng, up to about 1,550 ng, up to about 1,500 ng, up to about 1,450 ng, up to about 1,400 ng, up to about 1,350 ng, up to about 1,300 ng, up to about 1,250 ng, up to about 1,200 ng, up to about 1,150 ng, up to about 1,100 ng, up to about 1,050 ng, up to about 1,000 ng, up to about 950 ng, up to about 900 ng, up to about 850 ng, up to about 800 ng, up to about 750 ng, up to about 700 ng, up to about 650 ng, up to about 600 ng, up to about 550 ng, up to about 500 ng, up to about 450 ng, up to about 400 ng, up to about 350 ng, up to about 300 ng, up to about 250 ng, up to about 200 ng, up to about 150 ng, up to about 100 ng, up to about 50 ng or up to about 25 ng of the therapeutic agent release by the microparticle in a single dose or as a total daily dose. In certain embodiments, the subject can be administered from about 50 ng to about 1,000 ng of the therapeutic agent released by the microparticle in a single dose or a total daily dose. In certain embodiments, a subject can be administered about 1,000 ng of the therapeutic agent released by the microparticle in a single dose or as a total daily dose. In certain embodiments, a subject can be administered about 25 ng or more of the therapeutic agent released by the microparticle in a single dose or as a total daily dose.

In certain embodiments, the therapeutic agent can be released by the microparticle at a dose of about 0.05 μg/kg to about 100 μg/kg. In certain embodiments, a subject can be administered up to about 2,000 μg of the therapeutic agent released by the microparticle in a single dose or as a total daily dose. For example, but not by way of limitation, a subject can be administered up to about 1,950 μg, up to about 1,900 μg, up to about 1,850 μg, up to about 1,800 μg, up to about 1,750 μg, up to about 1,700 μg, up to about 1,650 μg, up to about 1,600 g, up to about 1,550 μg, up to about 1,500 μg, up to about 1,450 μg, up to about 1,400 μg, up to about 1,350 μg, up to about 1,300 μg, up to about 1,250 μg, up to about 1,200 μg, up to about 1,150 μg, up to about 1,100 μg, up to about 1,050 μg, up to about 1,000 μg, up to about 950 g, up to about 900 μg, up to about 850 μg, up to about 800 μg, up to about 750 μg, up to about 700 μg, up to about 650 μg, up to about 600 μg, up to about 550 μg, up to about 500 μg, up to about 450 μg, up to about 400 μg, up to about 350 μg, up to about 300 μg, up to about 250 μg, up to about 200 μg, up to about 150 μg, up to about 100 μg, up to about 50 μg or up to about g of the therapeutic agent release by the microparticle in a single dose or as a total daily dose. In certain embodiments, the subject can be administered from about 50 μg to about 1,000 g of the therapeutic agent released by the microparticle in a single dose or a total daily dose. In certain embodiments, a subject can be administered about 1,000 μg of the therapeutic agent released by the microparticle in a single dose or as a total daily dose. In certain embodiments, a subject can be administered about 25 μg or more of the therapeutic agent released by the microparticle in a single dose or as a total daily dose.

In certain embodiments, the therapeutic agent can be released by the microparticle at a dose of about 1 mg/kg to about 10 mg/kg. In certain embodiments, a subject can be administered up to about 20 mg of the therapeutic agent released by the microparticle in a single dose or as a total daily dose. For example, but not by way of limitation, a subject can be administered up to about 19 mg, up to about 18 mg, up to about 17 mg, up to about 16 mg, up to about 15 mg, up to about 14 mg, up to about 13 mg, up to about 12 mg, up to about 11 mg, up to about 10 mg, up to about 9 mg, up to about 8 mg, up to about 7 mg, up to about 6 mg, up to about 5 mg, up to about 4 mg, up to about 3 mg, up to about 2 mg or up to about 1 mg of the therapeutic agent release by the microparticle in a single dose or as a total daily dose. In certain embodiments, a subject can be administered about 10 mg or more of the therapeutic agent released by the microparticle in a single dose or as a total daily dose.

In certain embodiments, specific dosage regimes can be determined as described in Reagan-Shaw et al., FASEB J. 2008 March; 22(3):659-61, the content of which is incorporated by reference in its entirety.

It is to be understood that, for any particular subject, specific dosage regimes should be adjusted over time according to the individual need and the professional judgment of the person administering or supervising the administration of the microparticles. For example, the dosage of the microparticles can be increased if the lower dose does not provide sufficient activity in the treatment of a disease or condition described herein (e.g., inflammatory bowel disease). Alternatively, the dosage of the composition can be decreased if the disease (e.g., inflammatory bowel disease) is reduced, no longer detectable, or eliminated.

In certain embodiments, the microparticles can be administered once a day, twice a day, once a week, twice a week, three times a week, four times a week, five times a week, six times a week, once every two weeks, once a month, twice a month, once every other month or once every third month. In certain embodiments, the microparticles can be administered twice a week. In certain embodiments, the microparticles can be administered once a week. In certain embodiments, the microparticles can be administered two times a week for about four weeks and then administered once a week for the remaining duration of the treatment.

In certain embodiments, the period of treatment can be at least one day, at least one week, at least one month, at least two months, at least three months, at least four months, at least five months, or at least six months. In certain embodiments, the microparticles can be administered until the symptoms of inflammatory bowel disease are no longer detectable.

In certain embodiments, the microparticles can be administered to a subject by any route known in the art. In certain embodiments, the microparticles can be administered parenterally. In certain embodiments, the microparticles can be administered orally, intravenously, intraarterially, intrathecally, intranasally, subcutaneously, intramuscularly, and rectally. In certain embodiments, the microparticles can be administered orally. For example, but not by way of limitation, the present disclosure provides methods for the prevention and/or treatment of IBD in a subject by oral administration of the microparticles disclosed herein.

In certain embodiments, one or more microparticles can be used alone or in combination. For example, but not by way of limitation, methods of the present disclosure can include administering a first microparticle, e.g., including a TGF-β polypeptide, and a second microparticle, e.g., including an IL-2 polypeptide. In certain embodiments, methods of the present disclosure can include administering a first microparticle, e.g., including a TGF-β polypeptide, a second microparticle, e.g., including an IL-2 polypeptide, and a third microparticle, e.g., including rapamycin. In certain embodiments, the first, second, and/or third microparticles can be physically combined prior to administration, administered by the same route, or be administered over the same time frame. In certain embodiments, the first, second, and/or third microparticles are not physically combined prior to administration, administered by the same route, or are not administered over the same time frame.

In certain embodiments, methods of the present disclosure can include the use of at least one microparticle, at least two microparticles, or at least three microparticles. For example, but not by way of limitation, methods of the present disclosure can include administering at least one microparticle including a TGF-β polypeptide. Alternatively, the at least one microparticle can include an IL-2 polypeptide or macrolide. In certain embodiments, methods of the present disclosure can include administering at least two microparticles, e.g., a first microparticle including a TGF-β polypeptide and a second microparticle including an IL-2 polypeptide. Alternatively, the at least two microparticles can include a first microparticle including a TGF-β polypeptide and a second microparticle including a macrolide, or a first microparticle including an IL-2 polypeptide and a second microparticle including a macrolide. In certain embodiments, methods of the present disclosure can include administering at least three microparticles, e.g., a first microparticle including a TGF-β polypeptide, a second microparticle including an IL-2 polypeptide, and a third microparticle including a macrolide. In certain embodiments, a secondary treatment is administered before a microparticle. In certain embodiments, the secondary treatment is administered after a microparticle. In certain embodiments, the secondary treatment is administered simultaneously with a microparticle.

A “secondary treatment,” as used herein, can be any molecule, compound, chemical, or composition that has an anti-inflammatory effect and is provided and/or administered in addition to the microparticles described herein. Secondary treatments include, but are not limited to, anti-inflammatory, antibiotics, aminosalicylates, biologics interrupting inflammation pathways (e.g., Humira®), and corticosteroids.

In certain embodiments, administration of the microparticles to the subject has a therapeutic benefit. A “therapeutic benefit” as used herein, refers to one or more of a reduction in inflammation in intestine tissues and/or a reduction in symptoms of IBD.

### 5. Kits

The present disclosure provides kits for use in the disclosed methods. In certain embodiments, a kit can include a container that includes a microparticle or a pharmaceutical formulation thereof. In certain embodiments, the container can include a single dose of the microparticle or a pharmaceutical formulation thereof or multiple doses of the microparticle or a pharmaceutical formulation thereof. A container can be any receptacle and closure suitable for storing, shipping, dispensing, and/or handling a pharmaceutical product.

In certain embodiments, the kit can further include a second container that includes a solvent, carrier, and/or solution for diluting and/or resuspending the microparticle or a pharmaceutical formulation thereof. For example, but not by way of limitation, the second container can include sterile water.

In certain embodiments, the kits include a sterile container that contains the microparticle or a pharmaceutical formulation thereof; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In certain embodiments, the kit can further include instructions for administering the microparticle or a pharmaceutical formulation thereof. The instructions can include information about the use of the microparticle or a pharmaceutical formulation thereof for treating IBD. In certain embodiments, the instructions include at least one of the following: description of the microparticles; dosage schedule and administration for treating IBD; precautions; warnings; indications; counter-indications; overdosage information; adverse reactions; animal pharmacology; clinical studies; and/or references. The instructions can be printed directly on the container (when present), or as a label applied to the container, or as a separate sheet, pamphlet, card, or folder supplied in or with the container. For example, but not by way of limitation, the instructions can describe the method for administration and the dosage amount. In certain embodiments, the instructions indicate that the microparticles or pharmaceutical formulation thereof can be administered orally. In certain embodiments, the instructions can indicate that the microparticles or a pharmaceutical formulation thereof can be administered to a subject at a dose of between about 0.05 mg/kg to about 100 mg/kg.

In certain embodiments, the kit can further include a device for administering the microparticles or a pharmaceutical formulation thereof. For example, but not by way of limitation, the device can include a syringe, catheter, e.g., implantable catheter, and/or pump.

### EXAMPLES

The presently disclosed subject matter will be better understood by reference to the following Examples, which are provided as exemplary of the presently disclosed subject matter, and not by way of limitation.

### Example 1

Regulatory T cells are key mediators of tolerance in diseases of autoimmunity including in murine models of IBD and human IBD. The present example describes biomaterial strategies using microparticles including IL-2, TGF-beta, and rapamycin to enrich and program regulatory T cells in inflammatory bowel disease.

### Materials and Methods:

Network Analysis. To uncover the shared signaling between the IBD signaling network and TGF-beta, IL-2, and rapamycin, Ingenuity Pathway Analysis software (IPA, Qiagen) was used to construct the IBD signaling network and overlay TGF-beta, IL-2, and rapamycin signaling. Specifically, molecules related to IBD were derived from the IPA database. Chemical compounds, except for proteins, were then removed (trimmed) from the pathways. TGF-beta, IL-2, and rapamycin were then added to the network and the Path Explorer tool in the Ingenuity Pathway Analysis workbench was used to create connections between TGF-beta, IL-2, and rapamycin with the proteins involved in IBD pathogenesis. Overlapping signaling is noted as chords connecting TGF-beta, IL-2, and rapamycin with proteins found within the IBD signaling network.

Microparticle Fabrication and Characterization. Microparticles (MP) containing TGF-beta, IL-2, and rapamycin were synthesized using a water-in-oil-in-water or oil-in-water emulsion followed by a solvent evaporation technique (Ratay et al., Sci Rep 7, 17527-17527 (2017)). In brief, 200 mg of poly(lactide-co-glycolide) (PLG) was dissolved in 4 μmL of dichloromethane. The MP containing IL-2 and rapamycin utilized 50:50 lactide:glycolide carboxylic acid terminated PLG with a molecular weight (MW) between 7-17 kDa. In the TGF-beta preparations, 170 mg of 50:50 lactide:glycolide ester terminated PLG with an MW between 7-17 kDa and 30 mg of 50:50 lactide:glycolide mPEG-PLGA (PolySciTech, West Lafayette, IN) were used to generate the MP. All reagents were purchased from Sigma Aldrich (St. Louis, MO) or Thermo Fisher Scientific (Waltham, MA) unless otherwise specified. 5 μg of recombinant TGF-beta (human TGF-β from PeproTech, Rocky Hill, NJ) was dissolved in 200 μL of deionized water. Similarly, 5 μg of recombinant IL-2 (murine IL-2 from R&D Systems, Minneapolis, MN) was dissolved in 200 μL of phosphate-buffered saline. The protein solutions were then added to the bulk organic phase and sonicated at 25% amplitude for 10s (Active Motif, Carlsbad, CA) to create the water-in-oil emulsion. For the rapamycin MPs, 1 mg of rapamycin (Alfa Aesar, Ward Hill, MA) was added to 100 μL of dimethyl sulfoxide that was then added directly to the polymer solution. Blank, control MP, were synthesized with vehicle solvent alone. The water-in-oil emulsion or the organic solution was added to 60 μmL of 2% w/v poly (vinyl alcohol) (PVA, MW ˜25 kDa, 98% hydrolyzed, Polysciences, Warrington, PA) in deionized water or 51.6 μmM NaCl solution for IL-2 and homogenized (L4RT-1, Silverson, East Longmeadow MA) for 1 μminute at 3,000 rpm. The double or single emulsion was then decanted into an 80 μmL solution of 1% w/v PVA in DI water or (51.6 μmM NaCl for IL-2) and stirred at 600 rpm for 3 hours to facilitate DCM evaporation. To limit denaturation, homogenization and stirring for the TGF-β and IL-2 preparations were completed on ice. After 3 h of stirring, the microparticles (MPs) were collected by centrifugation (200 μg, 5 μmin, 4° C.) and washed 4 times with DI water before lyophilizing for 48-72 hours. The MP morphology and size distribution were determined using scanning electron microscopy (JSM-6335F, Peabody, MA). Drug release kinetics was determined by placing 5-10 mg of MP in 1 mL of either PBS with 1% w/v bovine serum albumin (TGF-0 and IL-2) or PBS with 0.02% v/v Tween-80 (rapamycin) and incubating at 37° C. with end-over-end rotation. Samples were collected at pre-defined intervals with solution replacement. Drug release was quantified by ELISA (R&D Systems, Minneapolis, MN) or using a microplate reader with absorbance at 278 nm.

TNBS Colitis. 2,4,6-Trinitrobenzene-1-sulfonic acid (TNBS) colitis was induced in 6-12 week female SJL/J mice (Bang et al., Current Protocols in Pharmacology 72, 5.58.51-55.58.42 (2016)). Baseline weight of the animals was recorded and 1.5 mg of TNBS in 150 μl of 50% EtOH was administered per rectum using a lubricated polyethylene catheter into isoflurane-anesthetized mice that underwent an overnight fast. Animals were subsequently held by the tail in a vertical position for 45 s and then placed back into their cage. Age-matched control animals received no treatment including isoflurane anesthesia. Animals that lost more than 25% of their starting weight or appeared lethargic were sacrificed. All animal procedures were conducted in accordance with an approved IACUC protocol.

MP Therapy. In the first set of feasibility studies, the disease was induced with TNBS on day 0 except in the case of the naïve control animals that did not undergo disease induction. On day 3 animals underwent no additional treatment (TNBS group) or were treated orally with 10 mg blank MP (Blank MP), a 150 μl bolus of soluble factors (Soluble Factors, 0.09 μg of TGF-β and IL-2 and 17 μg rapamycin, 100% theoretical) or 10 mg MP following encapsulation of 0.09 μg of TGF-β and IL-2 and 17 μg rapamycin. In subsequent studies a similar quantity of (the factors or) MP with or without the factors was administered however, an additional dose was added on the day following disease induction, day 1, for a total of 2 therapeutic treatments. For the oral and rectal routes, the microparticles were suspended in 150 μl and 100 μl of solvent, respectively. In particular, for the soluble factors, the samples were placed on ice to prevent loss of bioactivity. Statistical assessment of the survival curves was completed using the log-rank test (GraphPad Prism 8.2.1, GraphPad Software, San Diego, CA). The investigator was blinded to the groupings of the mice.

Histologic Scoring. The colon without mesentery was resected from the animals and washed with PBS. Following washing, the samples were blotted dry and the length and weight were obtained. Longitudinal sections of the colon were subsequently prepared following a Swiss roll method after fixation in formalin and paraffin-embedded. Histopathologic scoring was adapted from multiple sources (Bang et al., Current Protocols in Pharmacology 72, 5.58.51-55.58.42 (2016); Williams et al., Current protocols in mouse biology 6, 148-168 (2016); Kuemmerle et al., Methods in molecular biology 1422, 243-252 (2016)). Scoring was based upon the degree of inflammation and architectural distortion and could range from 0 (no disease) to 6 (severe disease). The pathologist was blinded to the group and not involved with disease induction, treatment, and animal monitoring (see Table 2).

TABLE 2
Histologic Scoring System

| Inflam- | | | | | | |
|---|---|---|---|---|---|---|
| mation | | | Architecture Score | | | |
| Score | | | Epithelial | Mucosal | | Total |
| Severity | Extent | Score | changes | architecture | Score | Score |
| Mild | Mucosa | 1 | Focal | | 1 | 0-6 |
| | | | erosions | | | |
| Moderate | Mucosa | 2 | Erosions | ±Focal | 2 | |
| | and sub- | | | ulcerations | | |
| | mucosa | | | | | |
| Marked | Trans- | 3 | | Extended | 3 | |
| | mural | | | ulcerations ± | | |
| | | | | granulation | | |
| | | | | tissue ± | | |
| | | | | pseudopolyps | | |

In silico and in vitro degradation of bioactive factors within the gastric environment. The in silico cytokine degradation was predicted using the Expasy PeptideCutter software package (E. Gasteiger et al., in The Proteomics Protocols Handbook, J. M. Walker, Ed. (Humana Press, Totowa, N J, 2005), pp. 571-607). In vitro degradation of IL-2 was measured in simulated gastric fluid (Ricca Chemical Company, Arlington, Texas) at pH 2.0. For the second set of the experimental conditions, pepsin (3.2 μg/L, Sigma) was also included. The samples were incubated for 100 μmin at 37° C. on a rocker and then neutralized before ELISA assay (R and D systems) for the soluble factors. For the MP containing factors, following treatment in the simulated gastric fluid the MPs were removed and washed multiple times with PBS supplemented with BSA and then incubated for 22 hours in PBS with 0.5% BSA. The released IL-2 was then quantitated with an ELISA excluding that which was released during the initial 100 μmin incubation in the simulated gastric fluid.

Colonic Adhesion. 3 days following TNBS colitis induction, the distal 2 cm of colon excluding the anus was washed and placed in ice-cold PBS. The colons were subsequently transferred to 500 ul solutions containing fluorescent microparticles (10 μm fluorescent PLG microspheres at a concentration of 10 mg/ml, Phosphorex Inc.) and incubated on a rocker at 37° C. for 30 μminutes. The samples were washed 5× and snap-frozen in liquid nitrogen, covered and later viewed using the IVIS imaging system (Lumina XR system, Caliper Life Sciences/PerkinElmer, Waltham, MA).

### Results:

It was previously demonstrated the utility of parenteral delivered microparticles containing IL-2, TGF-beta, and rapamycin for ameliorating diseases of immunity in preclinical models of transplant rejection, periodontal disease, inflammatory arthritis, and keratoconjunctivitis sicca. Thus, it was explored whether MP therapy could be useful when administered orally rather than parenterally, localize preferentially to inflamed tissue, and treat IBD in preclinical animal models.

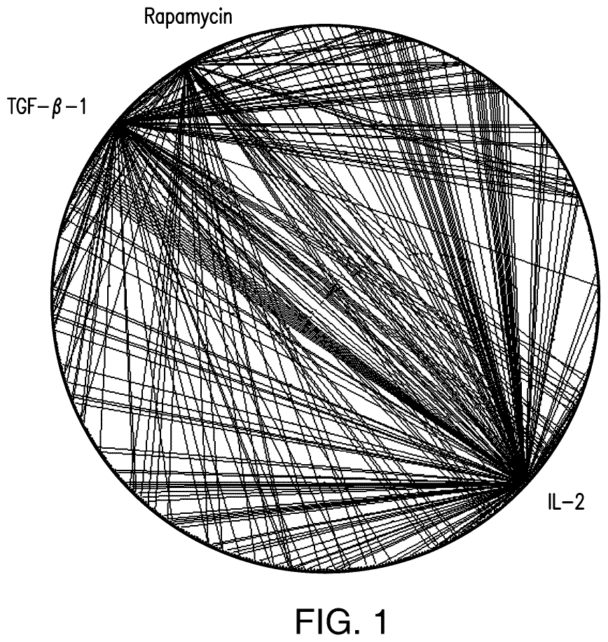

As a first step, to determine if IL-2, TGF-beta, and rapamycin could modulate the dysregulated protein network involved in IBD pathogenesis, proteins involved in IBD pathogenesis were mapped in silico to proteins affected by IL-2, TGF-beta, and rapamycin expression. As can be seen in FIG. 1, there was significant overlap in each of the factors and proteins implicated in IBD pathogenesis with the molecular networks of IL-2, TGF-beta, and rapamycin.

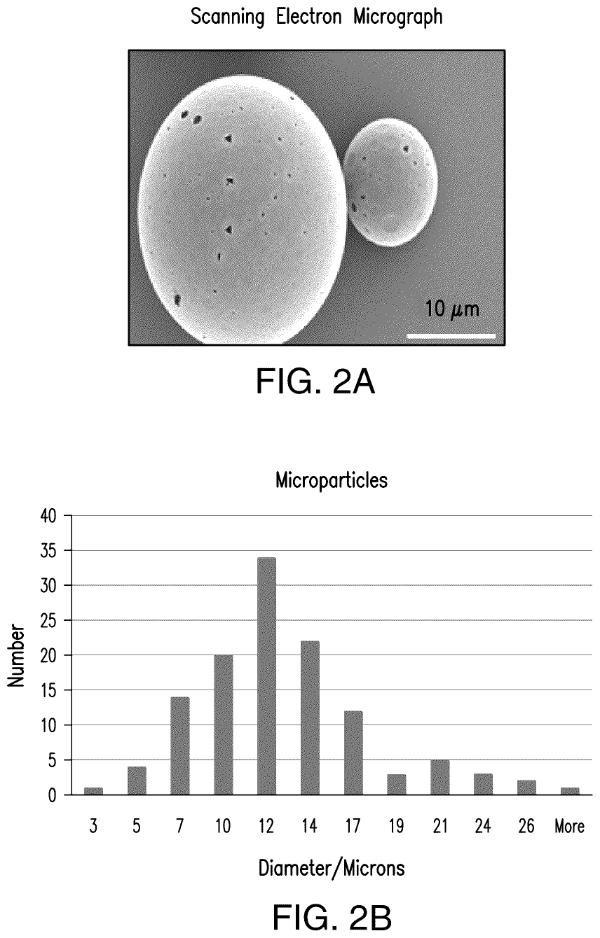

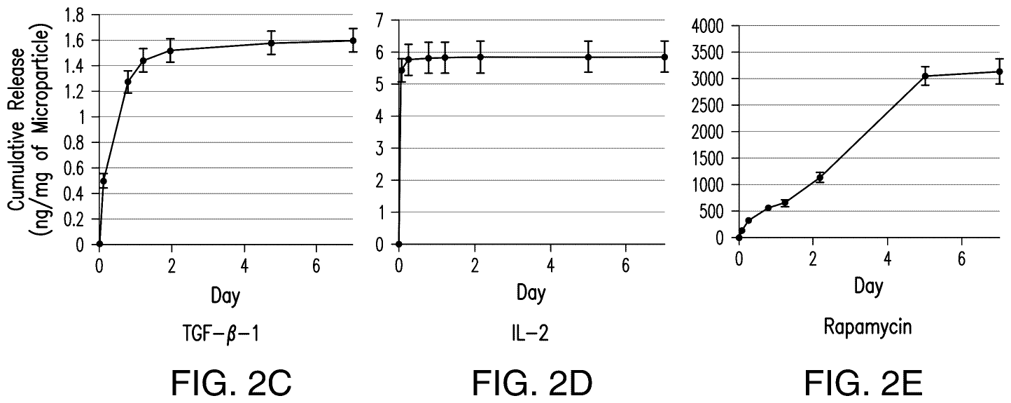

The next aim of this work was to design and build a microparticle system for delivering IL-2, rapamycin, and TGF-beta in a controlled fashion. Microparticles were synthesized using solvent evaporation techniques first creating oil-in-water (rapamycin) or water-in-oil-in-water emulsions (IL-2 and TGF-beta) for encapsulating drugs within poly(lactide-co-glycolide) microspheres, a polymer used within FDA approved therapies. Microparticles were synthesized with a mean diameter of 13+5 μm with a polydispersity index of 0.13 (see FIGS. 2A and 2B). Release of the factors could be observed for up to 1 week (see FIGS. 2C-2E).

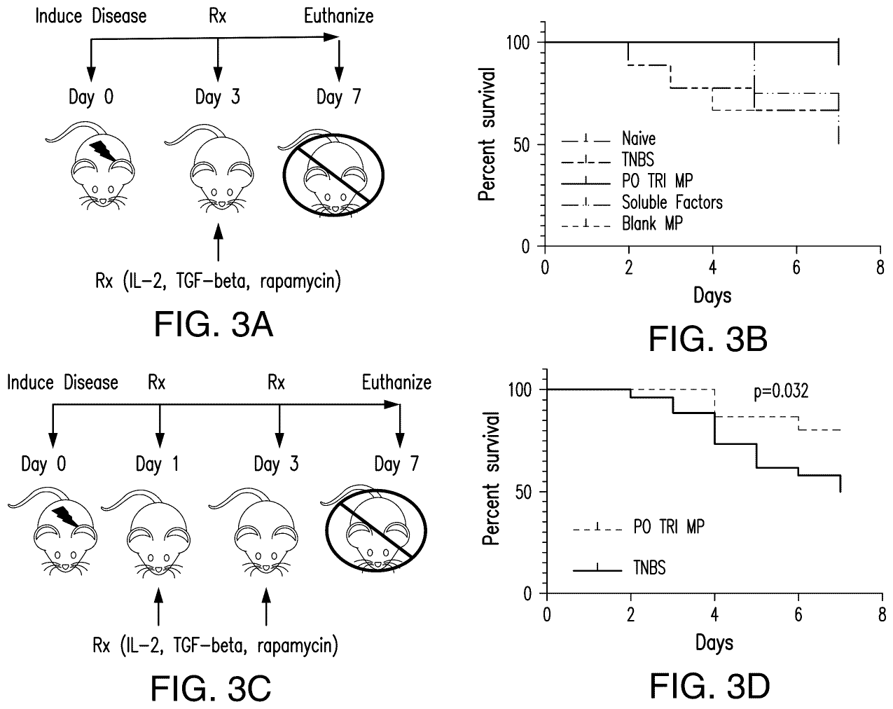

To explore whether or not the microparticle system could attenuate immunity in a preclinical model of inflammatory bowel disease, microparticles delivering the three factors or controls were administered to mice following induction of colitis in the murine TNBS colitis model. When a single administration of the microparticles was given three days after disease induction, there was a non-significant trend toward increased survival in the animals treated with microparticles given orally in comparison to untreated animals, blank MP, or a bolus of soluble factors (see FIGS. 3A and 3B). In particular, only 1 animal (13%) was sacrificed on day 7 in the group treated with tri-factor microparticles (TRI-MP) containing TGF-beta-1, IL-2, or rapamycin) in comparison to 3 animals (38%) that were sacrificed on days 2, 3, and 5 in the untreated group (TNBS).

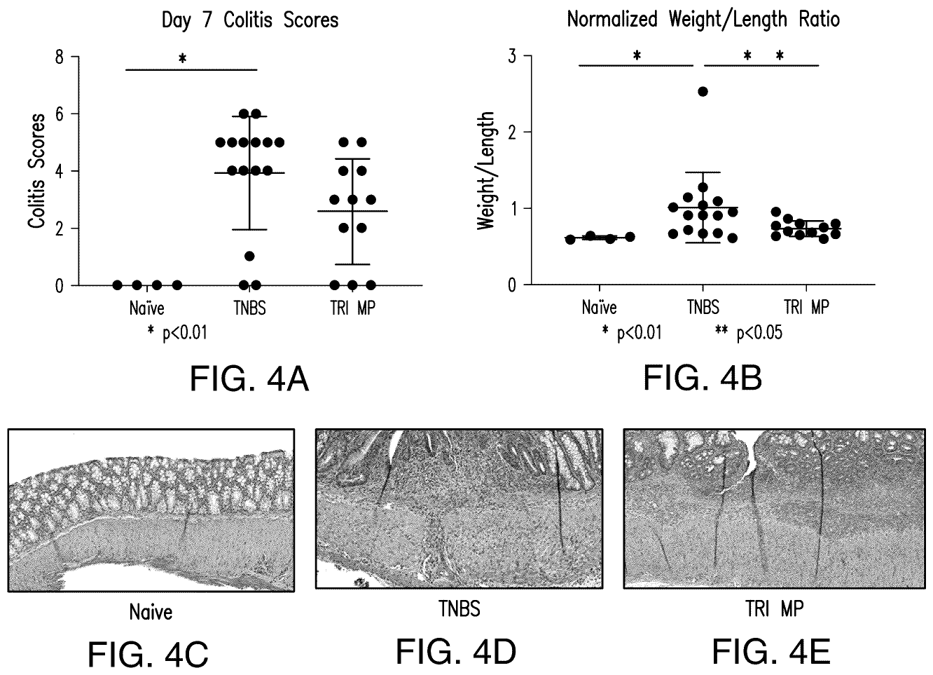

In the second set of experiments to attempt to increase potency (see FIGS. 3CA and 3D), following disease induction, mice were treated with microparticles on days 1 and 3 (PO TRI-MP) or left untreated (TNBS). The animals administered microparticles had a hazard ratio of 0.34 (0.12-0.90, 95% CI) with 80% of the animals surviving in the TRI-MP group and 50% in the untreated control group. Colons from animals with disease-induced 7 days prior that survived (or aged and sexed matched controls for the naïve control group) were measured and prepared for histology (see FIGS. 4A-4E). Colitis scores and weight/length ratios were significantly worse for the animals with disease-induced (TNBS) compared to naïve, matched controls. No difference was appreciated between the colitis scores and weight/length ratio for the TRI-MP treated group compared to the naïve mice. Also, the weight/length ratio was significantly improved for the TRI-MP-treated animals in comparison to the untreated controls (TNBS group).

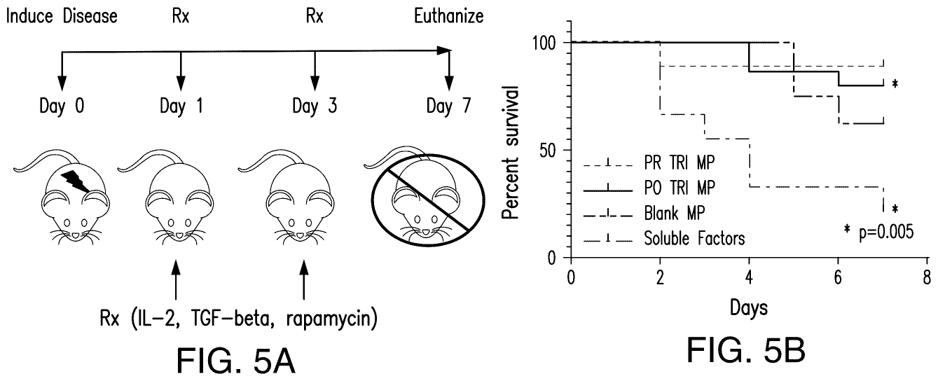

Given that TRI-MP could improve outcomes, it was investigated whether this was an effect of the formulation, not the vehicle or soluble factors alone and that the result could be re-capitulated with rectal administration. As before, the disease was induced on day 0, and on days 1 and 3 μmicroparticles or control formulations were administered. During the 7 days, there was no statistical difference between the TRI-MP administered rectally or orally groups with 89% and 80% (as before) animals surviving to 7 days, respectively. Although only 63% of animals survived in the blank, empty microparticle group, this was not statistically different than the TRI-MP cohort; however, soluble factors when administered as a bolus worsened outcome in comparison to the TRI-MP group with only 22% of animals surviving to day 7 with a corresponding hazard ratio of 6 (1.5 to 24, 95% CI). See FIGS. 5A and 5B.

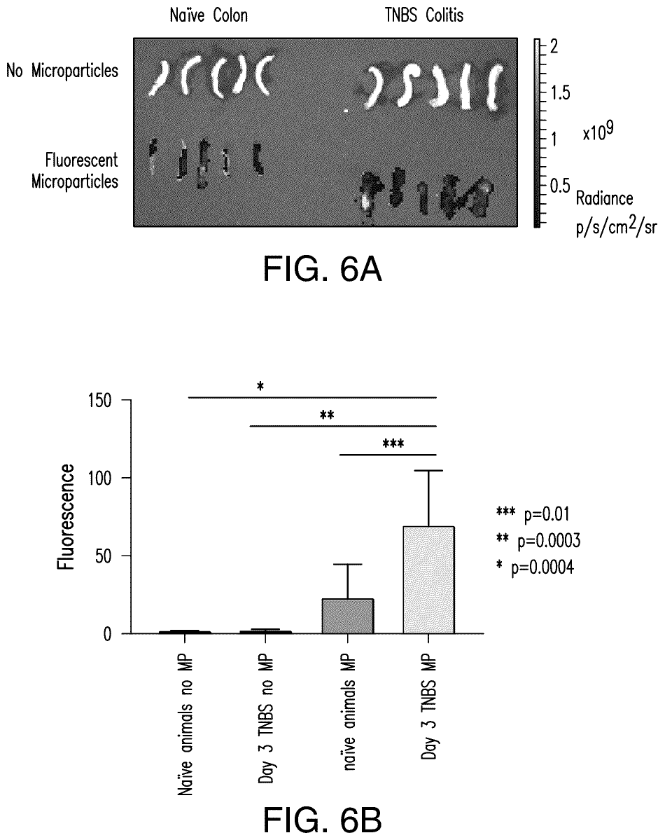

As a next step to better understand the mechanism for how the TRI-MPs were attenuating disease, localization of microparticles to healthy and inflamed colons was investigated. Colons were resected from either naïve mice or mice with colitis induced with TNBS (see FIGS. 6A and 6B). Fluorescently labeled microparticles were co-cultured with the colonic tissue and after extensive washing, the microparticles adhered much more prominently to the inflamed colons in comparison to the healthy colons.

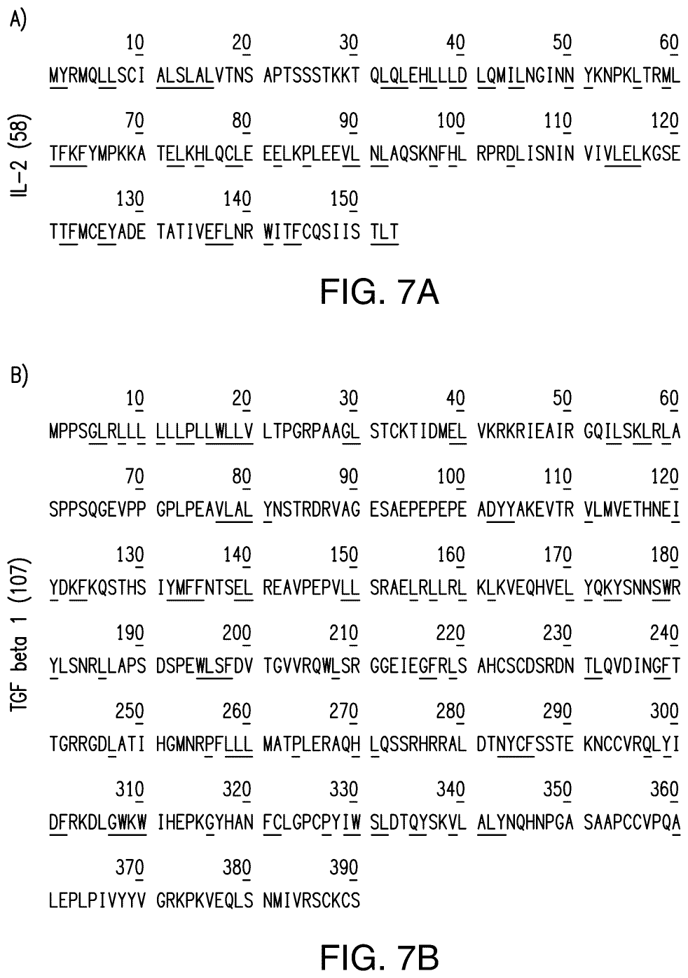

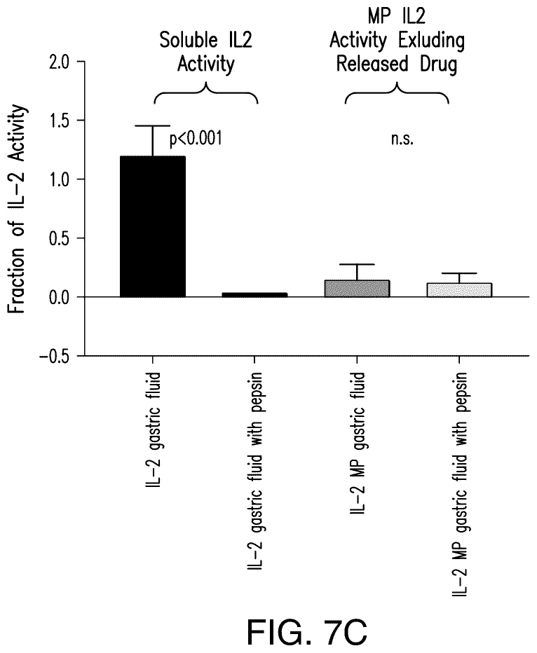

The microparticles preferentially adhered to inflamed colonic tissue however, the microparticles had to pass through the foregut before reaching the hindgut and during this process protected the protein cargo from degradation. In silico analysis showed that IL-2 and TGF-β-1 are extensively degraded by digestive enzymes when delivered without a carrier (see FIGS. 7A and 7B). These results were recapitulated in vitro for IL-2 with complete degradation when cultured in simulated gastric fluid containing pepsin (see FIG. 7C). On the other hand, nearly all of the IL-2 that is not delivered is detected by ELISA following microparticle culture in simulated gastric fluid with pepsin.

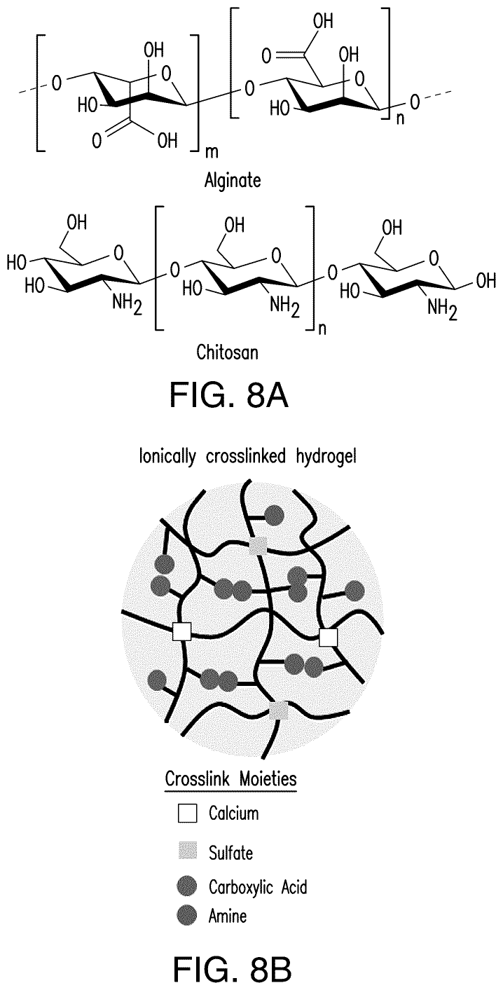

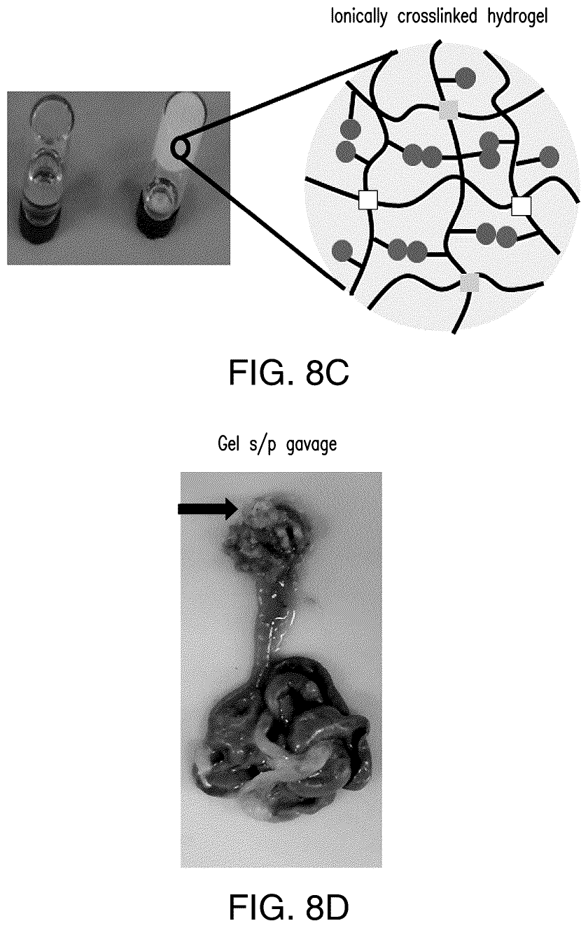

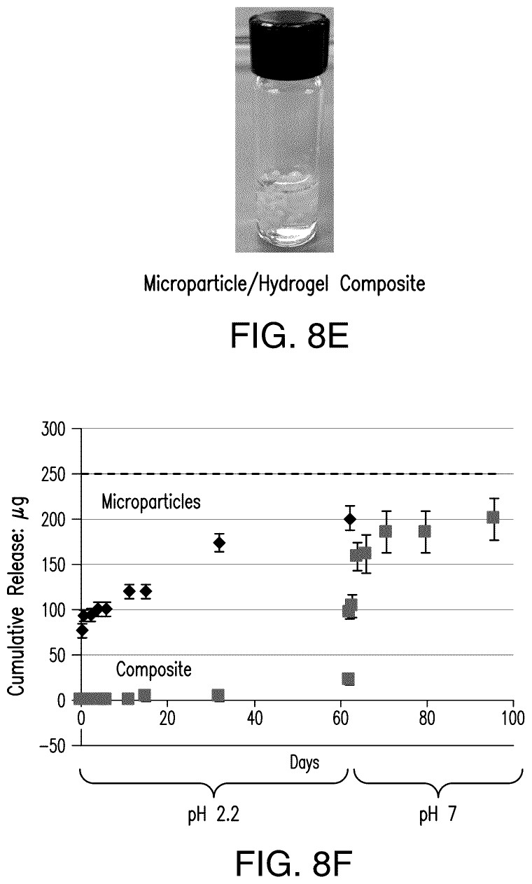

As the microparticles were able to attenuate colitis, possible biomaterial strategies to enhance the performance of the microparticle system were sought. As a first step toward this goal, alginate and chitosan ionic hydrogels were synthesized (see FIGS. 8A-8C). These microparticles can be embedded within the polymer and delivery of a model protein can be delayed until the pH approximating that of the intestine was achieved (see FIGS. 8D-8F).

### DISCUSSION

It was previously demonstrated that microparticles that deliver IL-2, TGF-beta, and rapamycin enrich regulatory T cells and through these cells promote local tolerance. Herein, the present example demonstrated that the microparticles can be administered orally, rather than parenterally, to attenuate ongoing colitis in a preclinical model of inflammatory bowel disease. The microparticles preferentially adhered to inflamed colonic tissue and the degradation of the factors in the gastric milieu was inhibited by the microparticles. Importantly, bolus delivery of the factors can worsen the overall disease course, suggesting the importance of controlled delivery.

Currently available therapies to treat inflammatory bowel disease non-specifically inhibit immune pathways systemically or target the colon in its entirety leading to off-target adverse effects, including increased incidences of infection and malignancy. Moreover, many of these therapies require parenteral administration. Thus, new strategies are necessary to target the gut locally and only in areas of disease with oral medications. The present example describes a first step toward achieving this goal by demonstrating a new platform for oral delivery of tolerogenic factors that localize preferentially to inflamed tissue and attenuate disease in an inflammatory bowel disease pre-clinical model.

Previous studies used ascorbyl palmitate hydrogels localized to inflamed colonic tissue via ionic interactions and delivering dexamethasone following enzymatic cleavage of the hydrogel (Zhang et al., Sci Transl Med 7, 300ra128-300ra128 (2015)); however, this approach required administration with an enema. Also, the mechanisms of dexamethasone-mediated immunosuppression are likely broader than that with the strategy reported here of coordinated delivery of multiple factors that have been previously shown to enrich regulatory T cells. Other studies utilized microparticle or microparticle carrier systems or even a combination of both (Nakase et al., The Journal of pharmacology and experimental therapeutics 292, 15-21 (2000); Naeem et al., Acta Biomaterialia 116, 368-382 (2020)).

The present disclosed microparticle-based strategy can also potentially limit the early burst release of drugs that is appreciated in many microparticle formulations. Nevertheless, the system could facilely be adapted to a microparticle formulation. As mentioned above, many of the microparticle studies deliver single factors, not coordinating the delivery of multiple factors in space and time to enrich for regulatory T cells to more specifically dampen immunity locally. A strength of the present example is that the approach has been shown to enhance regulatory T cell responses locally. Future studies examining the cellular mechanism for disease attenuation in the murine IBD model, particularly for regulatory and effector T cells, are warranted.

The present example describes a new strategy for treating IBD. Importantly, given the controlled release of the factors locally, orders of magnitude less drug were used in this study than would be used in traditional oral formulations. The PLG system designed and discussed in this example could be modified to incorporate antibody or peptide ligands to further enhance targeting. More generally, given the flexibility of the system, a wide variety of hydrophilic and hydrophobic pharmaceuticals from drugs that follow Lipinski's rule to peptides and siRNA can be delivered from this platform.

More broadly, diseases of inappropriate immune activation including eosinophilic esophagitis, celiac disease, microscopic colitis, many forms of hepatitis and pancreatitis and even possibly IBS are prevalent and carry significant morbidity. The platform described herein can be useful for treating many of these diseases. Further, in concert with engineering the cellular immune response, future iterations of this platform can allow for the re-direction of the microbiome toward health in combination allowing for new therapies for a broad range of disease indications.

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

What is claimed is:

1. A method for treating a gastrointestinal condition in a subject in need thereof comprising administering a composition comprising at least two of:
a) a microparticle comprising a transforming growth factor beta (TGF-β) polypeptide;
b) a microparticle comprising an interleukin polypeptide; or
c) a microparticle comprising a macrolide.

2. The method of claim 1, wherein the gastrointestinal condition is selected from the group consisting of inflammatory bowel disease (IBD), gastritis, peptic ulcers, oesophagitis, cholecystitis, Gastro-Intestinal Graft Versus Host Disease (GI-GVHD), gastrointestinal cancers or tumors, gastrointestinal infections, and gastrointestinal immunopathies.

3. The method of claim 1, wherein the microparticles are controlled release microparticles.

4. The method of claim 1, wherein the microparticles comprise a polymer.

5. The method of claim 4, wherein the polymer is selected from the group consisting of a poly(ethylene glycol) polymer, a poly(lactic acid) polymer, a poly(glycolic acid) polymer, a poly(lactide-co-glycolide) polymer, a polycaprolactone polymer, and a combination thereof.

6. The method of claim 1, wherein the microparticles have a diameter between about 5 μm to about 30 μm in diameter.

7. The method of claim 1, wherein the composition is a hydrogel composition.

8. The method of claim 7, wherein the hydrogel composition comprises a natural polymer selected from the group consisting of alginate, chitosan, and a combination thereof.

9. The method of claim 1, wherein the transforming growth factor beta (TGF-β) polypeptide is a TGF-β-1 polypeptide, wherein the interleukin polypeptide is IL-2, and wherein the macrolide is rapamycin.

10. The method of claim 1, wherein the TGF-β polypeptide comprises the amino acid sequence set forth in SEQ ID NO: 1 or SEQ ID NO:2.

11. The method of claim 10, wherein the TGF-β polypeptide comprises two copies of the amino acid sequence set forth in SEQ ID NO: 2.

12. The method of claim 11, wherein the two copies are linked by a disulfide bond.

13. The method of claim 1, wherein a weight ratio of the TGF-β polypeptide to the polymer of the microparticle is between about 1:100000 and about 1:1, wherein a weight ratio of the interleukin polypeptide to the polymer of the microparticle is between about 1:100000 and about 1:1, and wherein a weight ratio of the macrolide to the polymer of the microparticle is between about 1:100000 and about 1:1.

14. The method of claim 1, wherein the composition is administered orally, intravenously, or intranasally.

15. A composition for use in the treatment of a gastrointestinal condition, wherein the composition comprises at least two of:
a) a controlled release microparticle comprising a transforming growth factor beta (TGF-β) polypeptide;
b) a controlled release microparticle comprising an interleukin polypeptide; or
c) a controlled release microparticle comprising a macrolide.

16. The composition for use of claim 15, wherein the gastrointestinal condition is selected from the group consisting of inflammatory bowel disease (IBD), gastritis, peptic ulcers, esophagitis, cholecystitis, Gastro-Intestinal Graft Versus Host Disease (GI-GVHD), gastrointestinal cancers or tumors, gastrointestinal infections, and gastrointestinal immunopathies.

17. The composition for use of claim 15, wherein the controlled release microparticles comprise a polymer selected from the group consisting of a poly(ethylene glycol) polymer, a poly(lactic acid) polymer, a poly(glycolic acid) polymer, a poly(lactide-co-glycolide) polymer, a polycaprolactone polymer, and a combination thereof.

18. The composition for use of claim 15, wherein the composition is a hydrogel composition.

19. The composition for use of claim 15, wherein the transforming growth factor beta (TGF-β) polypeptide is a TGF-β-1 polypeptide, wherein the interleukin polypeptide is IL-2, and wherein the macrolide is rapamycin.

20. A kit for use in the treatment of a gastrointestinal condition, wherein the kit comprises at least two of:
a) a microparticle comprising a transforming growth factor beta (TGF-β) polypeptide;
b) a microparticle comprising an interleukin polypeptide; or
c) a microparticle comprising a macrolide.
